(19) United States
(12) Patent Application Publication
(10) Pub. No.: US 2008/0057057 A1
(43) Pub. Date: Mar. 6, 2008

(54) Anti-Lipid Rafts Antibodies

(75) Inventor: Kinsey Maundrell, Geneve (CH)

Correspondence Address: SALIWANCHIK LLOYD & SALIWANCHIK;A PROFESSIONAL ASSOCIATION, PO BOX 142950, GAINESVILLE, FL, 32614-2950, US

(73) Assignee: Applied Research Systems ARS Holding N.V., Curacao (NL)

(21) Appl. No.: 10/593,432

(22) PCT Filed: Mar. 18, 2005
(86) PCT No.: PCT/EP05/51267

§ 371 Date: Sep. 18, 2006

(30) Foreign Application Priority Data
Mar. 18, 2004 (EP) 04101129.7

(51) Int. Cl. A61K 39/395; A61K 39/00; A61P 25/28; A61P 7/12; C07K 16/18; C12N 5/06; G01N 33/00; G01N 33/53

(52) U.S. Cl. 424/133.1; 424/130.1; 424/141.1; 424/184.1; 435/332; 435/7.21; 436/86; 530/387.1; 530/387.3; 530/388.1

(57) ABSTRACT

The present invention relates to the discovery of antibodies able to modulate (prevent or favour) conversion of PrP^{c} to PrP^{Sc} and to their antigens. Depending on the nature of the antibodies (antagonistic or agonistic), their respective antigens are either conversion factors or inhibitors of prion replication.

### FIELD OF THE INVENTION

This invention relates to a method for generating anti-lipid rafts antibodies associated with a type of PrP^{Sc} cells (resistant or sensitive) as well as the hybridomas and antigens derived therefrom.

### BACKGROUND OF THE INVENTION

Creutzfeldt-Jakob disease (CJD) in humans and scrapie and bovine spongiform encephalopathy (BSE) in animals are some of the diseases that belong to the group of Transmissible Spongiform Encephalopathies (TSE), also known as prion diseases (Prusiner, 1991). These diseases are characterized by an extremely long incubation period, followed by a brief and invariably fatal clinical disease (Roos et al., 1973). To date no therapy is available.

Although these diseases are relatively rare in humans, the risk for the transmissibility of BSE to humans through the food chain has seized the attention of the public health authorities and the scientific community (Soto at al., 2001). Variant CJD (vCJD) is a new disease, which was first described in March 1996 (Will et al., 1996). In contrast to typical cases of sporadic CJD (sCJD), this variant form affects young patients (average age 27 years old) and has a relatively long duration of illness (median 14 months vs. 4.5 months in traditional CJD). A link between vCJD and BSE was first hypothesized because of the association of these two TSEs in place and time (Bruce, 2000). The most recent and powerful evidence comes from studies showing that the transmission characteristics of BSE and vCJD to mice are almost identical and strongly indicating that they are due to the same causative agent (Bruce et al., 1997). Moreover, transgenic mice carrying a human or a bovine gene have now been shown to be susceptible to BSE and vCJD (Scott et al., 1999). Furthermore, no other plausible hypothesis for the occurrence of vCJD has been proposed and intensive CJD surveillance in five European countries, with a low exposure to the BSE agent, has failed to identify any additional cases. In conclusion, the most likely cause of vCJD is exposure to the BSE agent, probably due to dietary contamination with affected bovine central nervous system tissue.

The nature of the transmissible agent has been matter of passionate controversy. Further research, has indicated that the TSE agent differs significantly from viruses and other conventional agents in that it seems not to contain nucleic acids (Prusiner, 1998). Additionally, the physicochemical procedures that inactivate most viruses, such as disrupting nucleic acids, have proved ineffective in decreasing the infectivity of the TSE pathogen. In contrast, the procedures that degrade protein have been found to inactivate the pathogen (Prusiner, 1991). Accordingly, the theory that proposes that the transmissible agent is neither a virus nor other previously known infectious agent, but rather an unconventional agent consisting only of a protein recently gained widespread acceptability (Prusiner, 1998). This new class of pathogen was called a “prion”, short for “proteinaceous infectious particle”. In TSE, prions are composed mainly of a misfolded protein named PrP^{Sc} (for scrapie PrP), which is a post-translationally modified version of a normal protein, termed PrP^{C} (Cohen et al., 1998). Chemical differences have not been detected to distinguish these two PrP isoforms and the conversion seems to involve a conformational change (FIG. 1) whereby the α-helical content of the normal protein diminishes and the amount of β-sheet increases (Pan et al., 1993). The structural changes are followed by alterations in the biochemical properties: PrP^{C} is soluble in non-denaturing detergents, PrP^{Sc} is insoluble; PrP^{C} is readily digested by proteases (also called protease sensitive prion protein) while PrP^{Sc} is partially resistant, resulting in the formation of a N-terminally truncated fragment known as PrPres (protease resistant prion protein) (Cohen et al., 1998).

The notion that endogenous PrP^{C} is involved in the development of infection is supported by experiments in mice in which the endogenous PrP gene was knocked out and where the animals were both resistant to prion disease and unable to generate new infectious particles (Bueler et al., 1993). In addition, it is clear that during the time between the inoculation with the infectious protein and the appearance of the clinical symptoms, there is a dramatic increase in the amount of PrP^{Sc}.

These findings suggest that endogenous PrP^{C} is converted to the PrP^{Sc} conformation by the action of an infectious form of the PrP molecule (Soto et al., 2001). Prion replication is hypothesized to occur when PrP^{Sc} in the infecting inoculum interacts specifically with host PrP^{C}, catalyzing its conversion to the pathogenic form of the protein. A physical association between the two isoforms during the infectious process is suggested by the primary sequence specificity in prion transmission (Telling et al., 1994) and by the reported in vitro generation of PrP^{Sc}-like molecules by mixing purified PrP^{C} with PrP^{Sc} (Saborio et al., 2001). However, the exact mechanism underlying the conversion is not known.

Investigations with chimeric transgenes showed that PrP^{C} and PrP^{Sc} are likely to interact within a central domain delimited by codons 96 and 169 (Prusiner, 1996) and synthetic PrP peptides spanning the region 109-141 proved to be able to bind to PrP^{C} and compete with PrP^{Sc} interaction (Chabry et al., 1998).

Based on data with transgenic animals, it has been proposed that additional brain factors present in the host are essential for prion propagation (Telling et al., 1995). It has been demonstrated previously that prion conversion does not occur under experimental conditions where purified PrP^{C} and PrP^{Sc} are mixed and incubated (Saborio et al., 1999) but that the conversion activity is recovered when the bulk of cellular proteins are added back to the sample (Saborio et al., 1999). This finding provides direct evidence that other factors present in the brain are essential to catalyze prion propagation.

The observation that cholesterol depletion decreases the formation of PrP^{Sc} whereas sphingolipid depletion increases PrP^{Sc} formation, suggested that “lipid rafts” (lipid domains in membranes that contain sphingolipids and cholesterol, see below) may be the site of the PrP^{c} to PrP^{Sc} conversion reaction involving either a raft-associated protein or selected raft lipids (Fantini et al., 2002). However, the role of lipid rafts in prion infectivity is still unclear.

Lipid rafts are regions on the plasma membrane that have a different composition of lipids than the surrounding plasma membrane. They are enriched in signalling molecules and can change their size and composition in response to intra- or extracellular stimuli (Simons, K., et al., Nature Reviews/Molecular Cell Biology: Vol. 1 pp 31-39 (2000)). This action favours specific protein-protein interactions, resulting in the activation of signalling cascades. The most important role of rafts at the cell surface is their function in signal transduction. It has been shown that growth factor receptors and sensor molecules migrate to lipid rafts after liga nd binding or cross-linking.

One approach to the treatment and prevention of prion diseases has been to develop agents for blocking the transformation of PrP^{c} into PrP^{Sc}. Some agents proposed were Congo red dye (U.S. Pat. No. 5,276,059), nerve growth peptides (U.S. Pat. No. 5,134,121), fragments of prion proteins (U.S. Pat. No. 6,355,610) and beta-sheet breaker peptides (U.S. Pat. No. 5,948,763) but it would be desirable to develop new methods for identifying and inhibiting the prion conversion factor(s).

Studies mainly led by Prusiner's group, postulated the existence of a chaperone-like molecule that may assist the conversion of PrP^{C} to PrP^{Sc} (4). This molecule is often referred as to “protein X”, “factor X” or “conversion factor”. One of the conversion factors implicated has been identified as Apolipoprotein B (see EP03101795.7).

Apolipoprotein B is the major protein component of the two known atherogenic lipoproteins, Low Density Lipoproteins (LDL) and remnants of triglyceride-rich lipoproteins and is a ligand for the LDL receptor (Segrest et al., 2001). Apolipoprotein B is known for its prominent role in cholesterol transport and plasma lipoprotein metabolism via LDL receptor interactions.

The present invention relates to the discovery of antibodies able to modulate (prevent or favour) conversion of PrP^{C} to PrP^{Sc} and to their antigens. Depending of the nature of the antibodies (antagonistic or agonistic), their respective antigens are either conversion factors or inhibitors of prion replication.

### SUMMARY OF THE INVENTION

In a first aspect, the present invention provides a method for generating an antibody against a lipid raft target associated with a type of PrP^{Sc} cells (i.e. resistant or sensitive PrP^{Sc} cells), comprising: isolating lipid rafts from said type of PrP^{Sc} cells; and immunizing an animal host by said lipid rafts.

The method according to the first aspect of the invention preferably further comprises: producing hybridomas from the immunized animal host, wherein said hybridomas produce monoclonal antibodies; selecting said monoclonal antibodies; and purifying said selected antibodies.

In a second aspect, the invention provides a method of identifying a lipid raft target comprising identifying an antigen that binds to the selected antibodies of the first preferred aspect of the invention, wherein said identifying comprises identifying a partial or full amino acid or nucleic acid of said antigen.

In a third aspect, the invention provides hybridomas according to the first aspect of the invention.

In a fourth aspect, the invention provides antibodies that bind to the isolated lipid raft according to the first aspect of the invention, wherein the antibodies modulate (e.g. prevents or favours) the conversion of PrP^{C} into PrP^{Sc}. The invention therefore also provides the monoclonal antibodies, antibodies or fragment thereof according to the fourth aspect of the invention.

In a fifth aspect, the invention relates to antigens or specific parts thereof according to the second aspect of the invention.

In a sixth aspect, the antibodies of the invention are further capable of regulating a biochemical activity of the antigen according to the fifth aspect of the invention.

In a seventh aspect, the antibodies of the invention are further capable of specifically detecting the antigen according to the fifth aspect of the invention.

In an eight aspect, the invention provides a pharmaceutical composition comprising a pharmaceutically acceptable carrier and, as an active ingredient, being capable of specifically binding an antibody according to the fourth aspect of the invention or an antigen according to the fifth aspect of the invention.

In an ninth aspect, the invention provides a pharmaceutical composition according to the eight aspect of the invention, wherein said antibody is further capable of regulating a biochemical activity of an antigen according to the fifth aspect of the invention.

In a tenth aspect, the invention provides a composition-of-matter comprising a substrate covalently attached to an antigen according to the fifth aspect of the invention for selectively capturing the antibody capable of specifically binding said antigen.

In an eleventh aspect, the invention relates to a method of treatment of a disease caused or aggravated by the activity of an antigen according to the fifth aspect of the invention (the antigen being preferably a conversion factor) comprising the administration of an antibody specifically binding said antigen and being capable of preventing the conversion of PrP^{C} into PrP^{Sc} according to the fourth aspect of the invention.

In a twelfth aspect, the invention relates to a method of treatment of a disease comprising the administration of an antigen according to the fifth aspect of the invention capable of preventing the conversion of PrP^{C} into PrP^{Sc}.

In a thirteenth aspect, the invention relates to the use of an antigen according to the fifth aspect of the invention (the antigen being preferably an inhibitor of prion replication) being capable of preventing the conversion of PrP^{C} into PrP^{Sc} in the manufacture of a medicament for the treatment of a disease.

In a fourteenth aspect, the invention relates to the use of an antibody according to the fourth aspect of the invention being capable of specifically binding the antigen according to the fifth aspect of the invention in the manufacture of a medicament for the treatment of a disease caused or aggravated by the activity of said antigen.

In a fifteenth aspect, the invention provides a device, comprising: a support surface; and an antibody according to the fourth aspect of the invention bound to the surface of the support, the antibody being characterized by an ability to modulate (e.g. prevent or favour) the conversion of PrP^{C} into PrP^{Sc}.

In a sixteenth aspect, the invention provides the antibody according to the fourth aspect of the invention, further characterized by the ability of said antibody to neutralize PrP^{Sc} infectivity (thus, to prevent conversion of PrP^{C} into PrP^{Sc}).

In a seventeenth aspect, the invention provides the antigen according to the fifth aspect of the invention, further characterized by the ability of said antigen to neutralize PrP^{Sc} infectivity.

In an eighteenth aspect, the invention relates to a method of determining PrP^{Sc} infection in a dead animal, comprising: extracting tissue from an animal that has died; contacting the tissue with an antibody according to the fourth aspect of the invention, wherein the antibody binds to the antigen according to the fifth aspect of the invention specific to the animal that has died; and determining if the antibody has bound to the antigen; wherein presence of the antigen in the tissue is indicative of PrP^{Sc} infection.

In a nineteenth aspect, the invention relates to a method of purifying a material suspected of containing the antigen according to the fifth aspect of the invention, comprising: contacting the material with a sufficient amount of an antibody characterized by its ability to bind the antigen in situ which antibody is bound to a support surface, and removing material not bound to the antibody.

In a twentieth aspect, the invention relates to the use of the antigen according to the fifth aspect of the invention or the antibody according to the fourth aspect of the invention in an assay (e.g. preferably Protein Misfolding Cyclic Amplification (PMCA) assay) for the detection of the formation of PrP^{sc} in a sample.

In a twentyfirst aspect, the invention relates to the use of the antigen according to the fifth aspect of the invention or the antibody according to the fourth aspect of the invention in a screening assay for identifying compounds that modulate the conversion of PrP^{c} into PrP^{sc}.

In a twentysecond aspect, the invention relates to the use of a modulator (e.g. the antibody according to the fourth aspect of the invention) of the antigen according to the fifth aspect of the invention for the preparation of a pharmaceutical preparation for the treatment of a prion disease.

In a twentythird aspect, the invention relates to the use of the antibody according to the fourth aspect of the invention for the preparation of a pharmaceutical formulation for the treatment of a conformational disease. Preferably, said antibody is able to prevent conversion of PrP^{c} into PrP^{sc}.

In a twentyfourth aspect, the invention relates to the use of the antigen according to the fifth aspect of the invention for the preparation of a pharmaceutical formulation for the treatment of a prion disease. Preferably, said antigen is an inhibitor of prion replication.

In a twentyfifth aspect, the invention relates to a method for the diagnosis or detection of a prion disease within a subject suspected of suffering from such a disease which comprises (i) obtaining a sample from the subject; (ii) contacting a sample from said subject with the antigen according to the fifth aspect of the invention or with the antibody according to the fourth aspect of the invention being able to favour conversion of PrP^{c} into PrP^{sc}; (iii) contacting the mixture obtained in step (ii) with PrP^{C} or PrP^{C} containing mixtures; and (iv) determining the presence and/or amount of PrP^{Sc} in said sample.

In a twentysixth aspect, the invention relates to a method for the diagnosis or detection of a prion disease within a subject suspected of suffering from such a disease which comprises (i) obtaining a sample from the subject; (ii) contacting a sample from said subject with the antigen according to the fifth aspect of the invention or with the antibody according to the fourth aspect of the invention being able to favour conversion of PrP^{c} into PrP^{sc} and at least another conversion factor (e.g. Apolipoprotein B or a fragment thereof); (iii) contacting the mixture obtained in step (ii) with PrP^{C} or PrP^{C} containing mixtures; and (iv) determining the presence and/or amount of PrP^{Sc} in said sample.

In a twentyseventh aspect, the invention provides a method of determining a marker that predisposes a subject to a prion disease, comprising (i) obtaining a sample from the subject; (ii) measuring a level of said antibody according to the fourth aspect of the invention or said antigen according to the fifth aspect of the invention; and (iii) correlating said level of protein obtained in said measuring step with the occurrence of a prion disease.

In a twentyeight aspect, the invention provides a method for the detection of PrP^{Sc} within a sample, which assay comprises (i) contacting said sample with said antibody according to the fourth aspect of the invention or with said antigen according to the fifth aspect of the invention; (ii) contacting sample obtained in (i) with PrP^{C} or PrP^{C} containing mixtures; and (iii) determining the presence and/or amount of PrP^{Sc} in said sample.

In a twentyninth aspect, the invention provides a method for the detection of PrP^{Sc} within a sample, which assay comprises (i) contacting said sample with said antibody according to the fourth aspect of the invention or with said antigen according to the fifth aspect of the invention and at least another conversion factor (e.g. Apolipoprotein B or a fragment thereof); (ii) contacting sample obtained in (i) with PrP^{C} or PrP^{C} containing mixtures; and (iii) determining the presence and/or amount of PrP^{Sc} in said sample.

In a thirtieth aspect, the invention provides a method for identifying a compound which modulates the transition of PrP^{C} into PrP^{Sc} comprising: (i) contacting said sample with the antigen according to the fifth aspect of the invention or with the antibody according the fourth aspect of the invention (a) in the presence of said modulatory compound and (b) in the absence of said compound; (ii) contacting the mixtures obtained in step (i) a and (i) b with PrP^{C} or PrP^{C} containing mixtures; and (iii) determining the amount of PrP^{Sc} (a) in the presence of said modulatory compound and (b) in the absence of said modulatory compound.

In a thirtyfirst aspect, the invention provides a method for identifying a compound which modulates the transition of PrP^{C} into PrP^{Sc} comprising: (i) contacting said sample with the antigen according to the fifth aspect of the invention or with the antibody according the fourth aspect of the invention and at least another conversion factor (e.g. Apolipoprotein B or a fragment thereof) (a) in the presence of said modulatory compound and (b) in the absence of said compound; (ii) contacting the mixtures obtained in step (i) a and (i) b with PrP^{C} or PrP^{C} containing mixtures; and (iii) determining the amount of PrP^{Sc} (a) in the presence of said modulatory compound and (b) in the absence of said modulatory compound.

In a thirtysecond aspect, the invention provides an assay for the detection of PrP^{Sc} in a sample within a sample, which assay comprises (i) contacting said sample with the antigen according to the fifth aspect of the invention or with the antibody according the fourth aspect of the invention; (ii) contacting the mixture obtained in step (i) with PrP^{C} or PrP^{C} containing mixtures; (iii) determining the presence and/or amount of PrP^{Sc} in said sample.

In a thirtythird aspect, the invention provides an assay for the detection of PrP^{Sc} in a sample within a sample, which assay comprises (i) contacting said sample with the antigen according to the fifth aspect of the invention, or with the antibody according the fourth aspect of the invention and at least another conversion factor and at least another conversion factor (e.g. Apolipoprotein B or a fragment thereof); (ii) contacting the mixture obtained in step (i) with PrP^{C} or PrP^{C} containing mixtures; (iii) determining the presence and/or amount of PrP^{Sc} in said sample.

In a thirtyfourth aspect, the invention provides a screening assay for identifying a compound which modulates the transition of PrP^{C} into PrP^{Sc} comprising: (i) contacting said sample with the antigen according to the fifth aspect of the invention or with the antibody according the fourth aspect of the invention (a) in the presence of said modulatory compound and (b) in the absence of said modulatory compound; (ii) contacting the mixtures obtained in step (i) a and (i) b with PrP^{C} or PrP^{C} containing mixtures; and (iii) determining the amount of PrP^{Sc} (a) in the presence of said compound and (b) in the absence of said modulatory compound.

In a thirtyfifth aspect, the invention provides a screening assay for identifying a compound which modulates the transition of PrP^{C} into PrP^{Sc} comprising: (i) contacting the antigen according to the fifth aspect of the invention, or with the antibody according the fourth aspect of the invention and at least another conversion factor (e.g. Apolipoprotein B or a fragment thereof) (a) in the presence of said modulatory compound and (b) in the absence of said modulatory compound; (ii) contacting the mixtures obtained in step (i) a and (i) b with PrP^{C} or PrP^{C} containing mixtures; and (iii) determining the amount of PrP^{Sc} (a) in the presence of said compound and (b) in the absence of said modulatory compound.

In a thirtysixth aspect, the invention provides a diagnostic kit for use in the assay according to any of the thirtyfifth, thirtysecond, thirtythird or thirthyfourth aspect of the invention, comprising a probe for receiving a sample and the antigen according to the fifth aspect of the invention or with the antibody according the fourth aspect of the invention.

In a thirtyseventh aspect, the invention provides a diagnostic kit for use in the assay according to any of the thirtyfifth, thirtysecond, thirtythird or thirthyfourth aspect of the invention, comprising a probe for receiving a sample and the antigen according to the fifth aspect of the invention or with the antibody according the fourth aspect of the invention and at least another conversion factor (e.g. Apolipoprotein B or a fragment thereof).

In a thirtyeight aspect, the invention provides an apparatus for use in the method of any of the preceding aspects or the assay of any of the preceding aspects.

### DESCRIPTION OF THE FIGURES

FIG. 1: Conformational change of the prion protein in prion-related diseases. The normal prion protein (PrP^{C}) undergoes a drastic change in its secondary structure leading to the formation of the pathological isoform (PrP^{Sc})

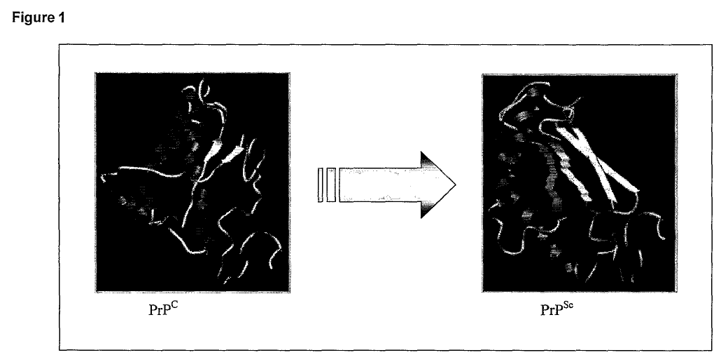

FIG. 2: Lipid rafts purification

FIG. 3: (A) Cell dot-blotting of resistant (#23) and sensitive (#60) subclones, prior and after scrapie infection and after 10 months of passaging. Both subclones display a stable phenotype. (B) PrP^{C} is expressed at similar levels in both subclones and is highly enriched in lipid rafts. Lane 1: total extract, lane 2: sample layer (40% sucrose), lane 3: lipid rafts layer (15% sucrose).

FIG. 4: ELISA with several dilutions of serum tested against lipid rafts. Immunisations clearly produced an immunogenic response reflected by an increase in the antibody titer

FIG. 5: (A) Sensitive cells may possess a conversion factor that directly promotes PrP^{C} conversion. This factor may be absent in resistant cells. (B) Alternatively, resistant cells may express an inhibitor that impairs PrP^{C} conversion by protecting it from converting molecules. Even though they are not illustrated in these models, interactions between PrP^{C} and PrP^{Sc} are also necessary for the conversion.

FIG. 6: Antibody interactions with lipid rafts were measured in function of known amounts of proteins. Anti-6H4 was used to define the threshold of detection because PrP^{C} is known for being enriched in these domains (blue). Total IgGs from a naive mouse were used as a negative control (purple).

FIG. 7: Primary screening of Mabs—ELISA—. This figure provides an example of how Mabs were selected. Mabs were tested against total lipid rafts from #23 and #60. Results were considered as “positives” (blue) when OD was above the negative control value and “negatives” (red) when below or similar values. Anti-FDC M2, a monoclonal rat antibody non-reactive with lipid rafts, was used as negative control.

FIG. 8: FACS histograms representing the number of cells in function of their fluorescence. Briefly, if a cell population is stained with a secondary antibody conjugated to phycoerythrin, a shift is observed. (A) #23 and #60 incubated with anti-6H4 (positive control, yellow curve). Several negative controls (blue curve), including HAT medium alone, were used and perfectly overlapped excluding any kind of artefact related to the composition of the selection medium. Both subclones displayed identical patterns. (B) Examples of two Mabs against lipids rafts tested with #23 and #60, both are positives but do not show any differential shift.

FIG. 9: Test of cell dot blotting in 96-well plates

FIG. 10: Screening of Mabs in the cell-based prion replication assay. Each Mab was tested in duplicate in two separate plates. Results were put next to each other to make easier the comparison. Some Mabs inhibit prion replication (red squares) whereas others inhibit cell growth (black squares). Controls: (HAT) cells cultured in complete DMEM/HAT medium 1:1, (DMEM) cells cultured in complete DMEM.

FIG. 11: This figure provides the final results of the effect of purified Mabs on PrP^{Sc} replication. To test the effect of Mabs on PrP replication, the prion-sensitive N2a subclone #60 was infected with the RML strain of PrP^{Sc} and grown for 4 passages in medium containing 2 ug/ml Mab. The positive control was the anti-prion antibody 6H4; the negative controls were Mabs #s 93, 122 and 306. The results shown are for Mabs for which cell growth was unaffected. The data show that 6H4 is a powerful inhibitor of PrP^{Sc} replication, confirming results already in the literature, and that the purified antibodies from the hybridomas previously defined as negative controls do not affect PrPSc replication. The positive Mabs are #s, 5, 51, 57, 197, and 245 for which PrP^{Sc} replication is inhibited.

### DETAILED DESCRIPTION OF THE INVENTION

The present invention relates to the discovery of antibodies able to modulate (prevent or favour) conversion of PrP^{C} to PrP^{Sc} and to their antigens. Depending of the nature of the antibodies, being either antagonists or agonists of PrP^{C} to PrP^{Sc} conversion, their respective antigens are either conversion factors or inhibitors of prion replication.

The following paragraphs provide definitions of various terms, and are intended to apply uniformly throughout the specification and claims unless an otherwise expressly set out definition provides a different definition.

The term “prion” shall mean a transmissible particle known to cause a group of such transmissible conformational diseases (spongiform encephalopathies) in humans and animals. The term “prion” is a contraction of the words “protein” and “infection” and the particles are comprised largely if not exclusively of PrP^{Sc} molecules.

“Prions” are distinct from bacteria, viruses and viroids. Known prions include those which infect animals to cause scrapie, a transmissible, degenerative disease of the nervous system of sheep and goats as well as bovine spongiform encephalopathies (BSE) or mad cow disease and feline spongiform encephalopathies of cats. Four prion diseases known to affect humans are Kuru, Creutzfeldt-Jakob Disease (CJD), Gerstmann-Strassler-Scheinker Disease (GSS), and fatal familial insomnia (FFI) (Prusinier, 1991). As used herein prion includes all forms of prions causing all or any of these diseases or others in any animals used—and in particular in humans and in domestic farm animals.

The terms “PrP protein”, “PrP” and the like are used interchangeably herein and shall mean both the infectious particle form PrP^{Sc} known to cause diseases (spongiform encephalopathies) in humans and animals and the non-infectious form PrP^{c} which, under appropriate conditions is converted to the infectious PrP^{Sc} form.

The term “PrP gene” refers generally to any gene of any species which encodes any form of a prion protein. Some commonly known PrP sequences are described in Gabriel et al., Proc. Natl. Acad. Sci. USA 89:9097-9101 (1992) which is incorporated herein by reference to disclose and describe such sequences. The PrP gene can be from any animal including the “host” and “test” animals described herein and any and all polymorphisms and mutations thereof, it being recognized that the terms include other such PrP genes that are yet to be discovered. The protein expressed by such a gene can assume either a PrP^{c} (non-disease) or PrP^{Sc} (disease) form.

The terms “standardized prion preparation”, “prion preparation”, “preparation” and the like are used interchangeably herein to describe a composition containing prions (PrP^{Sc}) which composition is obtained from brain tissue of mammals which contain substantially the same genetic material as relates to prions, e.g., brain tissue from a set of mammals which exhibit signs of prion disease which mammals (1) include a transgene as described herein; (2) have an ablated endogenous prion protein gene; (3) have a high copy number of prion protein gene from a genetically diverse species; or (4) are hybrids with an ablated endogenous prion protein gene and a prion protein gene from a genetically diverse species. The mammals from which standardized prion preparations are obtained exhibit clinical signs of CNS dysfunction as a result of inoculation with prions and/or due to developing the disease due to their genetically modified make up, e.g., high copy number of prion protein genes.

The term “artificial PrP gene” is used herein to encompass the term “chimeric PrP gene” as well as other recombinantly constructed genes which when included in the genome of a host animal (e.g., a mouse) will render the mammal susceptible to infection from prions which naturally only infect a genetically diverse test mammal, e.g., human, bovine or ovine. In general, an artificial gene will include the codon sequence of the PrP gene of the mammal being genetically altered with one or more (but not all, and generally less than 40) codons of the natural sequence being replaced with a different codon—preferably a corresponding codon of a genetically diverse mammal (such as a human). The genetically altered mammal being used to assay samples for prions which only infect the genetically diverse mammal. Artificial PrP genes can include not only codons of genetically diverse animals but may include codons and codon sequences not associated with any native PrP gene but which, when inserted into an animal render the animal susceptible to infection with prions which would normally only infect a genetically diverse animal.

The terms “chimeric gene,” “chimeric PrP gene”, “chimeric prion protein gene” and the like are used interchangeably herein to mean an artificially constructed gene containing the codons of a host animal such as a mouse with one or more of the codons being replaced with corresponding codons from a genetically diverse test animal such as a human, cow or sheep. In one specific example the chimeric gene is comprised of the starting and terminating sequence (i.e., N- and C-terminal codons) of a PrP gene of a mammal of a host species (e.g. a mouse) and also containing a nucleotide sequence of a corresponding portion of a PrP gene of a test mammal of a second species (e.g. a human). A chimeric gene will, when inserted into the genome of a mammal of the host species, render the mammal susceptible to infection with prions which normally infect only mammals of the second species. The preferred chimeric gene disclosed herein is MHu2M which contains the starting and terminating sequence of a mouse PrP gene and a non-terminal sequence region which is replaced with a corresponding human sequence which differs from a mouse PrP gene in a manner such that the protein expressed thereby differs at nine residues.

The term “genetic material related to prions” is intended to cover any genetic material which effects the ability of an animal to become infected with prions. Thus, the term encompasses any “PrP gene”, “artificial PrP gene”, “chimeric PrP gene” or “ablated PrP gene” which terms are defined herein as well as modification of such which effect the ability of an animal to become infected with prions. Standardized prion preparations are produced using animals which all have substantially the same genetic material related to prions so that all of the animals will become infected with the same type of prions and will exhibit signs of infection at about the same time.

The terms “host animal” and “host mammal” are used to describe animals which will have their genome genetically and artificially manipulated so as to include genetic material which is not naturally present within the animal. For example, host animals include mice, hamsters and rats which have their PrP gene ablated i.e., rendered inoperative. The host is inoculated with prion proteins to generate antibodies. The cells producing the antibodies are a source of genetic material for making a phage library. Other host animals may have a natural (PrP) gene or one which is altered by the insertion of an artificial gene or by the insertion of a native PrP gene of a genetically diverse test animal.

The terms “test animal” and “test mammal” are used to describe the animal which is genetically diverse from the host animal in terms of differences between the PrP gene of the host animal and the PrP gene of the test animal. The test animal may be any animal for which one wishes to run an assay test to determine whether a given sample contains prions with which the test animal would generally be susceptible to infection For example, the test animal may be a human, cow, sheep, pig, horse, cat, dog or chicken, and one may wish to determine whether a particular sample includes prions which would normally only infect the test animal.

The terms “genetically diverse animal” and “genetically diverse mammal” are used to describe an animal which includes a native PrP codon sequence of the host animal which differs from the genetically diverse test animal by 17 or more codons, preferably 20 or more codons, and most preferably 28-40 codons. Thus, a mouse PrP gene is genetically diverse with respect to the PrP gene of a human, cow or sheep, but is not genetically diverse with respect to the PrP gene of a hamster. The terms “ablated PrP protein gene”, “disrupted PrP gene”, and the like are used interchangeably herein to mean an endogenous PrP gene which has been altered (e.g., add and/or remove nucleotides) in a manner so as to render the gene inoperative. Examples of non-functional PrP genes and methods of making such are disclosed in Bueler, H., et al “Normal development of mice lacking the neuronal cell-surface PrP protein” Nature 356, 577-582 (1992) and Weisman (WO 93/10227). The methodology for ablating a gene is taught in Capecchi, Cell 51:503-512 (1987) all of which are incorporated herein by reference. Preferably both alleles of the genes are disrupted.

The terms “hybrid animal”, “transgenic hybrid animal” and the like are used interchangeably herein to mean an animal obtained from the cross-breeding of a first animal having an ablated endogenous prion protein gene with a second animal which includes either (1) a chimeric gene or artificial PrP gene or (2) a PrP gene from a genetically diverse animal. For example a hybrid mouse is obtained by cross-breeding a mouse with an ablated mouse gene with a mouse containing (1) human PrP genes (which may be present in high copy numbers) or (2) chimeric genes. The term hybrid includes any offspring of a hybrid including inbred offspring of two hybrids provided the resulting offspring is susceptible to infection with prions with normal infect only a genetically diverse species. A hybrid animal can be inoculated with prions and serve as a source of cells for the creation of hybridomas to make monoclonal antibodies of the invention.

The terms “susceptible to infection” and “susceptible to infection by prions” and the like are used interchangeably herein to describe a transgenic or hybrid test animal which develops a disease if inoculated with prions which would normally only infect a genetically diverse test animal. The terms are used to describe a transgenic or hybrid animal such as a transgenic mouse Tg (MHu2M) which, without the chimeric PrP gene, would not become infected with a human prion but with the chimeric gene is susceptible to infection with human prions.

The term “prion conversion factor” refers to a factor comprising proteins, lipids, enzymes or receptors that acts as a co-factor or auxiliary factor involved in the process of conversion of PrP^{C} into PrP^{Sc} and favours the onset and/or progression of the prion disease.

The terms “standardized prion preparation”, “prion preparation” and the like are used interchangeably herein to describe a composition containing prions which composition is obtained for example from brain tissue of mammals substantially the same genetic material as relates to PrP proteins, e.g. brain tissue from a set of mammals which exhibit signs or prion disease or for example a composition which is obtained from chronically prion infected cells.

The term “type of PrP^{Sc} cells” refers to cells that are either sensitive to infection by prions, referred to herein as “PrP^{Sc} sensitive cells”, or resistant to infection by prions, referred to herein as “PrP^{Sc} resistant cells”.

The term “non-PrP^{Sc} sensitive cells” refers to a type of cells which is not sensitive to infection by prions.

The term “non-PrP^{Sc} resistant cells” refers to a type of cells which is not resistant to infection by prions.

The terms “sensitive to infection”, “sensitive to prion infection” and the like are use for a material from a mammal, including cells, that can be infected with an amount and type of prion which would be expected to cause prion disease or symptoms.

By analogy, the terms “resistant to infection”, “resistant to prion infection” and the like are used for a material from a mammal, including cells which has the characteristic to be resistant when infected with an amount and type of prion which would be expected to cause prion disease or symptoms and remain uninfected even after several infective prion material inoculations.

The term “sample” refers to a biological extract from a mammal, including cell sample, body fluid, genetic material such as brain homogenate, cells, lipid rafts or purified peptides and proteins.

The term “incubation time” shall mean the time from inoculation of an animal with a prion until the time when the animal first develops detectable symptoms of disease resulting from infection, it also means the time from inoculation of material from a mammal, e.g. brain homogenate, cells, lipid rafts from cells, with prion until the time when the prion infection is detectable such as through the conversion of PrP^{C} into PrP^{Sc}. Several methods of detection of prion infection and PrP conversion are known by a person skilled in the art.

The term “fraction” refers to any fragment of the polypeptidic chain of the compound itself, alone or in combination with related molecules or residues bound to it, for example residues of sugars or phosphates, or aggregates of the original polypeptide or peptide. Such molecules can result also from other modifications which do not normally alter primary sequence, for example in vivo or in vitro chemical derivativization of peptides (acetylation or carboxylation), those made by modifying the pattern of phosphorylation (introduction of phosphotyrosine, phosphoserine, or phosphothreonine residues) or glycosylation (by exposing the peptide to enzymes which affect glycosylation e.g., mammalian glycosylating or deglycosylating enzymes) of a peptide during its synthesis and processing or in further processing steps.

The terms “modulator” or “modulatory compound” refer to molecules that modify the functions and/or properties (such as receptor binding, lipid affinity, enzyme interaction, structural arrangement, synthesis, metabolism) of the natural protein. “Modulators” of “modulatory compounds” include “agonists” and antagonists”. Modulators” include peptides, proteins or fragments thereof, peptidomimetics, organic compounds and antibodies.

The term “mimetics” refer to molecules that mimic the functions a nd/or properties (such as receptor binding, lipid affinity, enzyme interaction, structural arrangement, synthesis, metabolism) of a natural protein. These compounds have for example the property to either enhance a property of the natural protein (i e. to lead to the same activity when the compound is added to the natural protein as obtained with an increase in concentration in the natural protein) or to exhibit the same property as a natural protein (i.e. to lead to the same activity when the compound replaces the natural protein). “Mimetics” include peptides, proteins or fragments thereof, peptidomimetics and organic compounds. Examples of apolipoprotein E mimetics are described in US20020128175.

The terms “inhibitor” or “antagonist” refer to molecules that alter partially or impair the functions and/or properties (such as receptor binding, lipid affinity, enzyme interaction, structural arrangement, synthesis, secretion, metabolism) of the natural protein. “Inhibitors” or “antagonists” include peptides, proteins or fragments thereof, peptidomimetics, organic compounds and antibodies. Examples of Apolipoprotein B antibodies are described in Choi et al., 1997 and in Wang et al., 2000. Examples of Apolipoprotein E antibodies are described in Aizawa et al., 1997 and Yamada et al., 1997. Examples of Apolipoprotein antagonists can be antagonists that alter or impair the role of Apolipoproteins B or E in the cholesterol transport pathway. Examples of compounds that alter Apolipoprotein B secretion or synthesis are described in U.S. Pat. No. 6,369,075, U.S. Pat. No. 6,197,972, WO 03002533 and WO 03045921. Other “modulators” or “antagonists” can be modulators of the LDL receptor, preferably LDL-receptor antagonists such as anti-LDL receptor antibodies. Examples of monoclonal antibodies to the LDL receptor are given in WO 0168710.

The term “protein misfolding cyclic amplification assay” or “PMCA assay” is an assay for the diagnosis or detection of conformational diseases which comprises a cyclic amplification system to increase the levels of the pathogenic conformer such as described for example in WO 0204954.

The term “marker” for a disease refers to a biological parameter or value including a genetic character, inherited protein mutation(s), blood level of a protein or an enzyme that is different from the average value in a heterogeneous population of individuals and whose occurrence correlates with the occurrence of said disease with a statistical significance. A “marker” for a disease or condition is typically defined as a certain cut-off level of a said biological variable. A “marker” provides basis for determining the risk (probability of occurrence) of a disease in a subject.

The term “complex” includes the formation of an entity by the interaction of several molecules, several proteins, several peptides together or with a receptor. These interactions may be reversible and/or transient. These interactions may induce changes in the properties of the interacting molecules, proteins, peptides or receptors.

By “effective amount”, it is meant a concentration of peptide(s) that is capable of slowing down or inhibiting the formation of PrP^{Sc} deposits, or of dissolving preformed deposits. Such concentrations can be routinely determined by those of skill in the art. It will also be appreciated by those of skill in the art that the dosage may be dependent on the stability of the administered peptide. A less stable peptide may require administration in multiple doses.

The term “lipid raft” refers to a lipid raft or a portion thereof in a clustered state or a non-clustered state, including “lipid raft”, “clustered lipid rafts”, and “DRM”, each of which has been described in detail in Simons, K., et al., Nature Reviews/Molecular Cell Biology: Vol. 1 pp 31-39 (2000). In particular, “lipid raft” contains a given set of proteins that can change size and composition in response to intra- or extracellular stimuli. This favours specific protein-protein interactions, resulting in the activation of signally cascade. Sometimes, the lipid rafts may be clustered together. It has been reported that clustering is used both artificially and physiologically to trigger signalling cascades. DRMs (detergent-resistant membranes) are the rafts that remain insoluble after treatment on ice with detergents. They are believed to be non-native aggregated rafts. Hence, “lipid rafts” refers to small platforms, composed of sphingolipids and cholesterol in the outer exoplasmic layer, connected to Cholesterol in the inner cytoplasmic layer of the bilayer that have been reviewed recently (Simons et al., 2000). Lipid rafts can be isolated as they are insoluble in certain detergents such as triton X-100 at 4° C. Therefore, rafts can be purified as detergent-insoluble membranes (DIMs) or detergent-resistant membranes (DRMs) by ultracentrifugation on sucrose gradients. Rafts are enriched in GPI-anchored proteins, as well as proteins involved in signal transduction and intracellular trafficking. In neurons, lipid rafts act as platforms for the signal transduction initiated by several classes of neurotrophic factors.

The term “antibody” or “immunoglobulin” is intended to encompass both polyclonal and monoclonal antibodies. The preferred antibody is a monoclonal antibody reactive with the antigen. The term “antibody” is also intended to encompass mixtures of more than one antibody reactive with the antigen (e.g., a cocktail of different types of monoclonal antibodies reactive with the antigen). The term “antibody” is further intended to encompass whole antibodies, biologically functional fragments thereof, single-chain antibodies, and genetically altered antibodies such as chimeric antibodies comprising portions from more than one species, bifunctional antibodies, antibody conjugates, humanized and human antibodies. Biologically functional anti body fragments, which can also be used, are those peptide fragments derived from an antibody that are sufficient for binding to the antigen. Antibody as used herein is meant to include the entire antibody as well as any antibody fragments (e.g. F(ab′).sub.2, Fab′, Fab, Fv) capable of binding the epitope, antigen or antigenic fragment of interest.

By “purified antibody” is meant one which is sufficiently free of other proteins, carbohydrates, and lipids with which it is naturally associated. Such an antibody “preferentially binds” to lipid raft antigens of the present invention (or an antigenic fragment thereof), i.e., does not substantially recognize and bind to other antigenically unrelated molecules. A purified antibody of the invention is preferably immunoreactive with and immunospecific for a lipid raft antigen of specific species and more preferably immunospecific for a native human lipid raft antigen.

By “binds specifically” is meant high avidity and/or high affinity binding of an antibody to a specific polypeptide i.e., epitope of a lipid raft antigen. Antibody binding to its epitope on this specific polypeptide is preferably stronger than binding of the same antibody to any other epitope. Antibodies which bind specifically to a lipid raft antigen of interest may be capable of binding other polypeptides at a weak, yet detectable, level (e.g., 10% or less of the binding shown to the polypeptide of interest). Such weak binding, or background binding, is readily discernible from the specific antibody binding to the compound or polypeptide of interest, e.g. by use of appropriate controls.

The term “genetically altered antibodies” means antibodies wherein the amino acid sequence has been varied from that of a native antibody. Because of the relevance of recombinant DNA techniques to this invention, one need not be confined to the sequences of amino acids found in natural antibodies; antibodies can be redesigned to obtain desired characteristics. The possible variations are many and range from the changing of just one or a few amino acids to the complete redesign of, for example, the variable or constant region. Changes in the constant region will, in general, be made in order to improve or alter characteristics, such as complement fixation, interaction with membranes and other effector functions. Changes in the variable region will be made in order to improve the antigen binding characteristics.

The term “humanized antibody” or “humanized immunoglobulin” refers to an immunoglobulin comprising a human framework, at least one and preferably all complimentarity determining regions (CDRs) from a non-human antibody, and in which any constant region present is substantially identical to a human immunoglobulin constant region, i.e., at least about 85-90%, preferably at least 95% identical. Hence, all parts of a humanized immunoglobulin, except possibly the CDRs, are substantially identical to corresponding parts of one or more native human immunoglobulin sequences. See, e.g. Queen et al., U.S. Pat. Nos. 5,5301,101; 5,585,089; 5,693,762; and 6,180,370 (each of which is incorporated by reference in its entirety).

“Fully humanized antibodies” are molecules containing both the variable and constant region of the human immunoglobulin. Fully humanized antibodies can be potentially used for therapeutic use, where repeated treatments are required for chronic and relapsing diseases such as autoimmune diseases. One method for the preparation of fully human antibodies consist of “humanization” of the mouse humoral immune system, i.e. production of mouse strains able to produce human Ig (Xenomice), by the introduction of human immunoglobulin (Ig) loci into mice in which the endogenous Ig genes have been inactivated. The Ig loci are exceedingly complex in terms of both their physical structure and the gene rearrangement and expression processes required to ultimately produce a broad immune response. Antibody diversity is primarily generated by combinatorial rearrangement between different V, D, and J genes present in the Ig loci. These loci also contain the interspersed regulatory elements, which control antibody expression, allelic exclusion, class switching and affinity maturation. Introduction of unrearranged human 19 transgenes into mice has demonstrated that the mouse recombination machinery is compatible with human genes. Furthermore, hybridomas secreting antigen specific hu-mAbs of various isotypes can be obtained by Xenomice immunisation with antigen.

Fully humanized antibodies and methods for their production are known in the art (Mendez et al., Nature Genetics 15:146-156 (1997); Buggemann et al., Eur. J. Immunol. 21:1323-1326 (1991); Tomizuka et al., Proc. Natl. Acad. Sci. USA 97:722-727 (2000) Patent WO 98/24893.

The term “chimeric antibody” refers to an antibody in which the constant region comes from an antibody of one species (typically human) and the variable region comes from an antibody of another species (typically rodent). Hence, chimeric antibodies are molecules of which different portions are derived from different animal species, such as those having the variable region derived from a murine Mab and a human immunoglobulin constant region. Chimeric antibodies are primarily used to reduce immunogenicity in application and to increase yields in production, for example, where murine Mabs have higher yields from hybridomas but higher immunogenicity in humans, such that human/murine chimeric Mabs are used. Chimeric antibodies and methods for their production are known in the art (Cabilly et al., Proc. Natl. Acad. Sci. USA 81:3273-3277 (1984); Morrison et al., Proc. Natl. Acad. Sci. USA 81:6851-6855 (1984); Boulianne et al., Nature 312:643-646 (1984); Cabilly et al., European Patent Application 125023 (published Nov. 14, 1984); Neuberger et al., Nature 314:268-270 (1985); Taniguchi et al., European Patent Application 171496 (published Feb. 19, 1985); Morrison et al., European Patent Application 173494 (published Mar. 5, 1986); Neuberger et al., PCT Application WO 8601533, (published Mar. 13, 1986); Kudo et al., European Patent Application 184187 (published Jun. 11, 1986); Sahagan et al., J. Immunol. 137:1066-1074 (1986); Robinson et al., International Patent Application No. WO8702671 (published May 7, 1987); Liu et al., Proc. Natl. Acad. Sci USA 84:3439-3443 (1987); Sun et al., Proc. Natl. Acad. Sci USA 84:214-218 (1987); Better et al., Science 240:1041-1043 (1988); Riechmann et al., Nature 332:323-327. and Harlow and Lane, ANTIBODIES: A LABORATORY MANUAL, supra. These references are entirely incorporated herein by reference.

As used herein, the phrase “antibody fragment” refers to a molecule comprising a portion of an antibody capable of specifically binding an antigen, an antigenic determinant or an epitope. It will be appreciated that Fab and F(ab′)2 and other fragments of the antibodies useful in the present invention may be used for the detection and quantitation of their antigens according to the methods disclosed herein for intact antibody molecules. Such fragments are typically produced by proteolytic cleavage, using enzymes such as papain (to produce Fab fragments) or pepsin (to produce F(ab′)2 fragments).

As regards the antibodies mentioned herein throughout, the term “monoclonal antibody” is meant to include monoclonal antibodies, chimeric antibodies, fully humanized antibodies, antibodies to anti-idiotypic antibodies (anti-anti-Id antibody) that can be labeled in soluble or bound form, as well as fragments thereof provided by any known technique, such as, but not limited to enzymatic cleavage, peptide synthesis or recombinant techniques. A monoclonal antibody contains a substantially homogeneous population of antibodies specific to antigens, which populations contain substantially similar epitope binding sites. Mabs may be obtained by methods known to those skilled in the art. See, for example Kohler and Milstein, Nature, 256:495-497 (1975); U.S. Pat. No. 4,376,110; Ausubel et al., eds., Harlow and Lane ANTIBODIES: A LABORATORY MANUAL, Cold Spring Harbor Laboratory (1988); and Colligan et al., eds., Current Protocols in Immunology, Greene Publishing Assoc. and Wiley Interscience N.Y., (1992-1996), the contents of which references are incorporated entirely herein by reference. Such antibodies may be of any immunoglobulin class including IgG, IgM, IgE, IgA, GILD and any subclass thereof. A hybridoma producing a mAb of the present invention may be cultivated in vitro, in situ or in vivo. Production of high titers of Mabs in vivo or in situ makes this the presently preferred method of production. The term “monoclonal antibody” is also meant to include both intact molecules as well as fragments thereof, such as, for example, Fab and F(ab′)2, which are capable of binding antigen. Fab and F(ab′)2 fragments lack the Fc fragment of intact antibody, clear more rapidly from the circulation, and may have less non-specific tissue binding than an intact antibody (Wahl et al., J. Nucl. Med. 24:316-325 (1983)).

An anti-idiotypic (anti-Id) antibody is an antibody which recognizes unique determinants generally associated with the antigen-binding site of an antibody. An Id antibody can be prepared by immunizing an animal of the same species and genetic type (e.g. mouse strain) as the source of the Mab to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880, which is herein entirely incorporated by reference. The anti-Id antibody may also be used as an “immunogen” to induce an immune response in yet another animal, producing a so-called anti-anti-Id antibody. The anti-anti-Id may be epitopically identical to the original Mab, which induced the anti-Id. Thus, by using antibodies to the idiotypic determinants of a Mab, it is possible to identify other clones expressing antibodies of identical specificity. Accordingly, Mabs generated against anti-lipid rafts may be used to induce anti-Id antibodies in suitable animals, such as BALB/c mice. Spleen cells from such immunized mice are used to produce anti-Id hybridomas secreting anti-Id Mabs. Further, the anti-Id Mabs can be coupled to a carrier such as keyhole limpet hemocyanin (KLH) and used to immunize additional BALB/c mice. Sera from these mice will contain anti-anti-Id antibodies that have the binding properties of the original Mab specific for an epitope. The anti-Id Mabs thus have their own idiotypic epitopes, or “idiotopes” structurally similar to the epitope being evaluated.

A monoclonal antibody is said to be “capable of binding” a molecule if it is capable of specifically reacting with the molecule to thereby bind the molecule to the antibody.

The term “epitope” is meant to refer to that portion of any molecule capable of being bound by an antibody, which can also be recognized by that antibody. Epitopes or “antigenic determinants” usually consist of chemically active surface groupings of molecules such as amino acids or sugar side chains and have specific three dimensional structural characteristics as well as specific charge characteristics.

An “antigen” is a molecule or a portion of a molecule capable of being bound by an antibody, which antigen is additionally capable of inducing an animal to produce antibody capable of binding to an epitope of that antigen. An antigen may have one or more than one epitope. The specific reaction referred to above is meant to indicate that the antigen will react, in a highly selective manner, with an epitope on its corresponding antibody and not with the multitude of other antibodies which may be evoked by other antigens.

The antibodies, including fragments of antibodies, useful in the present invention may be used to quantitatively or qualitatively detect their antigens in a sample or to detect presence of cells that express their antigens. This can be accomplished by immunofluorescence techniques employing a fluorescently labeled antibody (see below) coupled with fluorescence microscopy, flow cytometric, or fluorometric detection.

The antibodies (or fragments thereof) useful in the present invention may be employed histologically, as in immunofluorescence or immunoelectron microscopy, for in situ detection of their antigens. In situ detection may be accomplished by removing a histological specimen from a patient, and providing the labeled antibody of the present invention to such a specimen. The antibody (or fragment) is preferably provided by applying or by overlaying the labeled antibody (or fragment) to a biological sample. Through the use of such a procedure, it is possible to determine not only the presence of the antigens but also its distribution on the examined tissue. Using the present invention, those of ordinary skill will readily perceive that any of wide variety of histological methods (such as staining procedures) can be modified in order to achieve such in situ detection.

Such assays for the antigens typically comprises incubating a biological sample, such as a biological fluid, a tissue extract, freshly harvested cells such as lymphocytes or leukocytes, or cells which have been incubated in tissue culture, in the presence of a labeled antibody capable of identifying the antigens, and detecting the antibody by any of a number of techniques well known in the art.

The biological sample may be coupled to a solid phase support or carrier such as nitrocellulose, or other solid support or carrier which is capable of immobilizing cells, cell particles or soluble proteins. The support or carrier may then be washed with suitable buffers followed by treatment with a labeled antibody in accordance with the present invention, as noted above. The solid phase support or carrier may then be washed with the buffer a second time to remove unbound antibody. The amount of bound label on said solid support or carrier may then be detected by conventional means.

By “solid phase support”, “solid phase carrier”, “solid support”, “solid carrier”, “support” or “carrier” is intended any support or carrier capable of binding antigen or antibodies. Well-known supports or carriers, include glass, polystyrene, polypropylene, polyethylene, dextran, nylon amylases, natural and modified celluloses, polyacrylamides, gabbros and magnetite. The nature of the carrier can be either soluble to some extent or insoluble for the purposes of the present invention. The support material may have virtually any possible structural configuration so long as the coupled molecule is capable of binding to an antigen or antibody. Thus, the support or carrier configuration may be spherical, as in a bead, cylindrical, as in the inside surface of a test tube, or the external surface of a rod. Alternatively, the surface may be flat such as a sheet, test strip, etc. Preferred supports or carriers include polystyrene beads. Those skilled in the art will know may other suitable carriers for binding antibody or antigen, or will be able to ascertain the same by use of routine experimentation.

The binding activity of a given lot of antibody, of the invention as noted above, may be determined according to well-known methods. Those skilled in the art will be able to determine operative and optimal assay conditions for each determination by employing routine experimentation.

Other such steps as washing, stirring, shaking, filtering and the like may be added to the assays as is customary or necessary for the particular situation.

One of the ways in which an antibody in accordance with the present invention can be labeled is by linking the same to an enzyme and used in an enzyme immunoassay (EIA). This enzyme, in turn, when later exposed to an appropriate substrate, will react with the substrate in such a manner as to produce a chemical moiety which can be detected, for example, by spectrophotometric, fluorometric or by visual means. Enzymes which can be used to detectably label the antibody include, but are not limited to, malate dehydrogenase, staphylococcal nuclease, delta-5-steroid isomeras, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase and acetylcholin-esterase. The detection can be accomplished by colorimetric methods which employ a chromogenic substrate for the enzyme. Detection may also be accomplished by visual comparison of the extent of enzymatic reaction of a substrate in comparison with similarly prepared standards.

Detection may be accomplished using any of a variety of other immunoassays. For example, by radioactive labeling the antibodies or antibody fragments, it is possible to detect R-PTPase through the use of a radioimmunoassay (RIA). A good description of RIA may be found in Laboratory Techniques and Biochemistry in Molecular Biology, by Work, T. S. et al., North Holland Publishing Company, NY (1978) with particular reference to the chapter entitled “An Introduction to Radioimmune Assay and Related Techniques” by Chard, T., incorporated by reference herein. The radioactive isotope can be detected by such means as the use of a g counter or a scintillation counter or by autoradiography.

It is also possible to label an antibody in accordance with the present invention with a fluorescent compound. When the fluorescently labeled antibody is exposed to light of the proper wavelength, its presence can be then detected due to fluorescence. Among the most commonly used fluorescent labeling compounds are fluorescein isothiocyanate, rhodamine, phycoerythrine, pycocyanin, allophycocyanin, o-phthaldehyde and fluorescamine.

The antibody can also be detectably labeled using fluorescence emitting metals such as ¹⁵²E, or others of the lanthanide series. These metals can be attached to the antibody using such metal chelating groups as diethylenetriamine pentaacetic acid (ETPA).

The antibody can also be detectably labeled by coupling it to a chemiluminescent compound. The presence of the chemiluminescent-tagged antibody is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

Likewise, a bioluminescent compound may be used to label the antibody of the present invention. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

An antibody molecule of the present invention may be adapted for utilization in an immunometric assay, also known as a “two-site” or “sandwich” assay. In a typical immunometric assay, a quantity of unlabeled antibody (or fragment of antibody) is bound to a solid support or carrier and a quantity of detectably labeled soluble antibody is added to permit detection and/or quantitation of the ternary complex formed between solid-phase antibody, antigen, and labeled antibody.

Typical, and preferred, immunometric assays include “forward” assays in which the antibody bound to the solid phase is first contacted with the sample being tested to extract the antigen from the sample by formation of a binary solid phase antibody-antigen complex. After a suitable incubation period, the solid support or carrier is washed to remove the residue of the fluid sample, including unreacted antigen, if any, and then contacted with the solution containing an unknown quantity of labeled antibody (which functions as a “reporter molecule”). After a second incubation period to permit the labeled antibody to complex with the antigen bound to the solid support or carrier through the unlabeled antibody, the solid support or carrier is washed a second time to remove the unreacted labeled antibody.

In another type of “sandwich” assay, which may also be useful with the antigens of the present invention, the so-called “simultaneous” and “reverse” assays are used. A simultaneous assay involves a single incubation step as the antibody bound to the solid support or carrier and labeled antibody are both added to the sample being tested at the same time. After the incubation is completed, the solid support or carrier is washed to remove the residue of fluid sample and uncomplexed labeled antibody. The presence of labeled antibody associated with the solid support or carrier is then determined, as it would be in a conventional “forward” sandwich assay.

In the “reverse” assay, stepwise addition first of a solution of labeled antibody to the fluid sample followed by the addition of unlabeled antibody bound to a solid support or carrier after a suitable incubation period is utilized. After a second incubation, the solid phase is washed in conventional fashion to free it of the residue of the sample being tested and the solution of unreacted labeled antibody. The determination of labeled antibody associated with a solid support or carrier is then determined as in the “simultaneous” and “forward” assays.

The antibodies of the invention can be used in connection with immunoaffinity chromatography technology. More specifically, the antibodies can be placed on the surface of a material within a chromatography column. Thereafter, a composition to be purified can be passed through the column. If the sample to be purified includes any lipid raft antigens which binds to the antibodies those lipid raft antigens wi 11 be removed from the sample and thereby purified.

Hence, in summary methods of diagnosis can be performed in vitro using a cellular sample (e.g., blood sample, lymph node biopsy or tissue) from a mammal or can be performed by in vivo imaging.

Compositions comprising the antibodies of the present invention can be used to detect the presence of a lipid raft target in a type of PrP^{Sc} sensitive cells, for example, by radioimmunoassay, ELISA, FACS, etc. One or more labeling moieties can be attached to the humanized immunoglobulin. Exemplary labeling moieties include radiopaque dyes, radiocontrast agents, fluorescent molecules, spin-labeled molecules, enzymes, or other labeling moieties of diagnostic value, particularly in radiologic or magnetic resonance imaging techniques.

The terms “conformationally altered protein”, “disease related conformation of a protein” and the like are used interchangeably here to describe any protein which has a three dimensional conformation associated with a disease. The conformation ally altered protein may cause the disease, be a factor in a symptom of the disease or appear as a result of other factors associated with the disease. The conformationally altered protein appears in another conformation which has the same amino acid sequence. In general, the conformationally altered protein formed is “constricted” in conformation as compared to the other “relaxed” conformation which is not associated with disease. Those skilled in the art reading this disclosure will recognize the applicability of the antibody formulations of the invention to other conformationally altered proteins even though the invention is described in general as regards to prions. The following is a non-limiting list of diseases with associated proteins which assemble two or more different conformations wherein at least one conformation is an example of a conformationally altered protein.

Disease Insoluble Proteins (disease is indicated first, followed by the insoluble protein)
Alzheimer's Disease APP, Ap peptide, al-antichymotrypsin, tau, non-Ap component, presenillin 1, presenillin 2, apoE
Prion diseases, Creutzfeldt Jakob disease, PrP scrapie and bovine spongiform encephalopathy
ALS SOD and neurofilament
Pick's disease Pick body
Parkinson's disease a-synuclein in Lewy bodies
Frontotemporal dementia tau in fibrils
Diabetes Type II Amylin
Multiple myeloma—IgGL-chain
Plasma cell dyscrasias
Familial amyloidotic polyneuropathy Transthyretin
Medullary carcinoma of thyroid Procalcitonin
Chronic renal failure 32-microglobulin
Congestive heart failure Atrial natriuretic factor
Senile cardiac and systemic amyloidosis Transthyretin
Chronic inflammation Serum Amyloid A
Atherosclerosis ApoA1
Familial amyloidosis Gelsolin
Huntington's disease Huntington

The term “Alzheimer's disease” (abbreviated herein as “AD”) as used herein refers to a condition associated with formation of neuritic plaques comprising amyloid p protein, primarily in the hippocampus and cerebral cortex, as well as impairment in both learning and memory. “AD” as used herein is meant to encompass both AD as well as AD-type pathologies.

The term “Gerstmann-Strassler-Scheinker Disease” abbreviated as “GSS” refers to a form of inherited human prion disease. The disease occurs from an autosomal dominant disorder. Family members who inherit the mutant gene succumb to GSS.

The term “AD-type pathology” as used herein refers to a combination of CNS alterations including, but not limited to, formation of neuritic plaques containing amyloid protein in the hippocampus and cerebral cortex. Such AD-type pathologies can include, but are not necessarily limited to, disorders associated with aberrant expression and/or deposition of APP, overexpression of APP, expression of aberrant APP gene products, and other phenomena associated with AD. Exemplary AD-type pathologies include, but are not necessarily limited to, AD-type pathologies associated with Down's syndrome that is associated with overexpression of APP.

The term “phenomenon associated with Alzheimer's disease” as used herein refers to a structural, molecular, or functional event associated with AD, particularly such an event that is readily assessable in an animal model. Such events include, but are not limited to, amyloid deposition, neuropathological developments, learning and memory deficits, and other AD-associated characteristics.

The term “cerebral amyloid angiopathy” (abbreviated herein as CAA) as used herein refers to a condition associated with formation of amyloid deposition within cerebral vessels which can be complicated by cerebral parenchymal hemorrhage. CAA is also associated with increased risk of stroke as well as development of cerebellar and subarachnoid haemorrhages (Winters (1987) Stroke 18: 311-324; Haan et al. (1994) Dementia 5: 210-213; Itoh et al. (1993) J. Neural. Sci. 116:135-414). CAA can also be associated with dementia prior to onset of haemorrhages. The vascular amyloid deposits associated with CAA can exist in the absence of AD, but are more frequently associated with AD.

The term “phenomenon associated with cerebral amyloid angiopathy” as used herein refers to a molecular, structural, or functional event associated with CAA, particularly such an event that is readily assessable in an animal model. Such events include, but are not limited to, amyloid deposition, cerebral parenchymal hemorrhage, and other CAA-associated characteristics.

The term “-amyloid deposit” as used herein refers to a deposit in the brain composed of Ap as well as other substances.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes:
(a) preventing the disease from occurring in a subject which may be predisposed to the disease but has not yet been diagnosed as having it;
(b) inhibiting the disease, i.e., arresting its development; or
(c) relieving the disease, i.e., causing regression of the disease. The invention is directed toward treating patients with infectious prions and is particularly directed toward treating humans infected with PrP^{Sc}, resulting in a disease of the central nervous system such as bovine spongiform encephalopathy; Creutzfeldt-Jakob Disease; fatal familial insomnia or Gerstmann-Strassler-Scheinker Disease.

By “a pharmaceutically effective” amount of a drug or pharmacologically active agent or pharmaceutical formulation is meant a nontoxic but sufficient amount of the drug, agent or formulation to provide the desired effect.

A “subject,” “individual” or “patient” is used interchangeably herein, which refers to a vertebrate, preferably a mammal, more preferably a human.

As used herein, the phrase “pharmaceutical composition” refers to a preparation of one or more of the active ingredients described herein with other chemical components such as physiologically suitable carriers and excipients. The purpose of a pharmaceutical composition is to facilitate administration of active ingredients to an organism.

Herein the term “active ingredients” refers to the antibody or antibody fragment of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered active ingredients. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

### Lipid Raft Immunization

The lipid rafts can be isolated by the methods known in the art, such as the method described in Green et al, J. Cell Biol. 146, 673-682 (1999). In particular, cells are lysed and added to a sucrose solution to form a sucrose step-gradient. The gradients are then centrifuged, and the lipid rafts float to a lighter fraction of the gradients. That fraction is then isolated and concentrated.

The present invention provides for a method of identifying anti-lipid raft antibodies, lipid is raft targets or lipid raft antigens by lipid raft immunization. Lipid raft immunization produces monoclonal antibodies against lipid rafts derived from a type of PrP^{Sc} cells (being either PrP^{Sc} sensitive cells or PrP^{Sc} resistant cells). Such monoclonal antibodies can be directly used in the treatment of conformational diseases after the verification of their anti-conformational disease activities. The antigens that bind to such monoclonal antibodies are then identified.

The present invention provides for a method for identifying anti-lipid raft antibodies such as antibodies against a lipid raft target associated with a conformational disease comprising isolating lipid rafts from said type of PrP^{Sc} cells; immunizing an animal with the isolated lipid rafts. Lipid raft preparation from PrP^{Sc} cells may be injected into an appropriate host animal, such as cow, horse, goat, rat, sheep, mouse, hamster, or macaque monkey, etc. The immunization may be boosted by multiple sequential injections.

Preferably, such a method further comprises: producing hybridomas from the immunized animal host, wherein said hybridomas produce monoclonal antibodies; selecting the hybridoma (monoclonal) antibodies; and purifying and identifying the hybridoma (monoclonal) antibodies.

In one embodiment of the present invention, after the immunization, the animal may be sacrificed and the lymphocytes of said animal may be elicited. The lymphocytes can produce or be capable of producing antibodies that specifically bind to the protein used for immunization. Lymphocytes then are fused with myeloma cells using suitable fusing agents to form hybridomas cells. Examples of myeloma cell lines include, but are not limited to NS0. The hybridomas cells may be seeded and grow in suitable culture medium in 96-well culture plate with a density of one hybridoma cell per well. More preferably, nucleic acid encoding an inhibitor of apoptosis may be delivered into the myeloma cells to prevent the B-cell death induced by the production of auto-antigens. Said nucleic acids Include, but are not limited to, anti-apoptosis genes, such as BCL-2. The experimental details of creating hybridomas cells are described in the Examples of the present invention.

Preferably, the anti-conformational disease agent may be identified by selecting hybridoma antibodies based on their differential binding reactivity to the type of PrP cells of interest. Hybridoma antibodies that bind to the type of PrP^{Sc} sensitive cells but not to PrP^{Sc} resistant cells or to non-PrP^{Sc} sensitive cells as well as hybridoma antibodies that bind to the type of PrP^{Sc} resistant cells but not to PrP^{Sc} sensitive cells or to non-PrP^{Sc} resistant cells may be selected for further study.

Preferably, the method of identifying anti-conformational disease agents by lipid raft immunization comprises purifying and identifying the hybridoma antibodies. In other words, the method comprises purifying and identifying the antibodies produced by the hybridomas and the antigens that bind to the antibody. The molecular weight of the antigens can be determined by immunoprecipitation experiments. The antigens and antibodies of the selected hybridomas can be further purified by affinity chromatography and the antigen identified by microsequencing or by mass spectrometry. The experimental procedures of immunoprecipitation, affinity chromatography, and microsequencing are known in prior art. In addition, the anti-conformational disease agents can be selected based on their ability to modulate (prevent or favour) the process involved in conformationally altered proteins.

The antibody produced by hybridomas can be directly used as an anti-conformational disease agent or anti-prion disease agent. The anti-conformational disease activity or anti-prion disease activity of the antibodies produced by hybridomas can be verified by cell proliferation assay, xenograft model, and cell adhesion and migration assay, but preferably by FACS and most preferably by the cell based prion replication assay described in the example. The experimental details are described in the Examples of the present application.

The method of identifying anti-lipid raft targets by lipid raft immunization comprises identifying the antigens that bind to the antibodies produced by hybridomas. The identity of the antigen can lead to the discovery of a group of potential conformational disease agents, anti-prion disease agents or conversion factors. The examples for those conformational disease agents or anti-prion disease agents include, but are not limited to, a molecule inhibiting, preventing or interfering with the change in the conformation of a protein, preferably inhibiting, preventing or interfering with the change of conformation of a non-pathogenic form of a protein to its pathogenic form, and more preferably preventing, inhibiting or interfering with the conversion of PrP^{c} to PrP^{Sc}, but also neutralizing the activities of said protein, a molecule down-regulating the expression of said protein, the molecule down-regulating the transcription of DNA encoding said protein, or anti-sense nucleic acid sequence of partial or full nucleic acid sequence encoding said protein.

The present invention provides an isolated lipid raft derived form any PrP^{Sc} cell, preferably from a PrP^{Sc} sensitive or resistant cell. Preferably said isolated lipid raft is clustered with other lipid rafts derived from said PrP^{Sc} sensitive or resistant cell. More preferably, said isolated lipid raft is a detergent resistant membrane (DRM).

The present invention provides a monoclonal antibody that binds to an isolated lipid rafts, preferably an isolated lipid raft derived from a PrP^{Sc} cell (resistant or sensitive), more preferably, said isolated lipid raft comprises a polypeptide that is differentially expressed in a type of PrP^{Sc} cell. Preferably, said monoclonal antibody is an isolated monoclonal antibody.

Typically, the monoclonal antibody binds to both isolated lipid raft and the polypeptide that is a component of the isolated lipid raft and differentially expressed in the PrP^{Sc} sensitive or resistant cell where the lipid raft is derived from. Preferably, the monoclonal antibody binds to an exposed epitope of the polypeptide. The term “exposed epitope” refers to an epitope of said polypeptide that is on the surface of the lipid raft comprising said polypeptide, and not concealed due to the association of the polypeptide with the lipid raft. Thus, said antibody binds both to the lipid raft and said polypeptide. Preferably, said polypeptide is differentially expressed in PrP^{Sc} sensitive or resistant cell.

The present invention provides compounds capable of controlling, including increasing and/or inhibiting, the conversion of PrP^{C} into PrP^{Sc} in prion diseases.

The activity of the compounds of the invention in controlling the conversion of PrP^{C} into PrP^{Sc} in prion diseases can be detected using, for example, an in vitro assay, such as that described by Saborio et al., 2001 which measures the ability of compounds of the invention to modulate the conversion of PrP^{C} into PrP^{Sc}.

The invention is now described by its different aspects and by its preferred methods or procedures.

In a first aspect, the present invention provides a method for generating an antibody against a lipid raft target associated with a type of PrP^{Sc} cells, comprising: isolating lipid rafts from said type of PrP^{Sc} cells; and immunizing an animal host by said lipid rafts.

Preferably, the type of PrP^{Sc} cells refers to PrP^{Sc} sensitive cells or to PrP^{Sc} resistant cells

The method according to the first aspect of the invention preferably further comprises: producing hybridomas from the immunized animal host, wherein said hybridomas produce monoclonal antibodies; selecting said monoclonal antibodies; and purifying said selected antibodies.

Preferably, said selecting comprises selecting monoclonal antibodies that bind to said type of PrP^{Sc} sensitive cells but not to PrP^{Sc} resistant cells or to non-PrP^{Sc} sensitive cells.

Preferably, said selecting comprises selecting monoclonal antibodies that bind to said type of PrP^{Sc} resistant cells but not to PrP^{Sc} sensitive cells or to non-PrP^{Sc} resistant cells.

Most preferably, said selecting further comprises selecting monoclonal antibodies that modulate conversion of PrP^{C} into PrP^{Sc} of said type of PrP^{Sc} sensitive cells.

Even most preferably, said selecting further comprises selecting monoclonal antibodies that prevent conversion of PrP^{C} into PrP^{Sc} of said type of PrP^{Sc} sensitive cells.

Even most preferably, said selecting further comprises selecting monoclonal antibodies that favour conversion of PrP^{C} into PrP^{Sc} of said type of PrP^{Sc} sensitive cells.

Preferably, said type of PrP^{Sc} sensitive cells according to the first aspect of the invention are neuroblastoma cells.

More preferably, said type of neuroblastoma cells are scN2A cells.

Even more preferably, said type of neuroblastoma cells are N2A cells.

Preferably, said PrP^{Sc} sensitive cells are designated #60 and said PrP^{Sc} resistant cells are designated #23 (see example 1).

In a second aspect, the invention provides a method of identifying a lipid raft target comprising identifying an antigen that binds to the selected antibodies of the first preferred aspect of the invention, wherein said Identifying comprises identifying a partial or full amino acid or nucleic acid of said antigen.

In a third aspect, the invention provides hybridomas according to the first aspect of the invention.

Preferably, the invention provides hybridomas that allows selection of antibodies able to modulate conversion of PrP^{C} into PrP^{Sc}.

In a fourth aspect, the invention provides antibodies that bind to the isolated lipid raft according to the first aspect of the invention, wherein the antibodies modulate (e.g. prevents or favours) the conversion of PrP^{C} into PrP^{Sc}. The invention therefore also provides the monoclonal antibodies, antibodies or fragment thereof according to the fourth aspect of the invention.

In a fifth aspect, the invention relates to antigens or specific parts thereof according to the second aspect of the invention.

Preferably, the invention provides antigens able to modulate the conversion of PrP^{C} into PrP^{Sc}. These antigens can be identified with the antibodies according to the fourth aspect of the invention.

Preferably, the invention provides hybridomas derived from neuroblastoma cells. The invention also provides the monoclonal antibodies, antibodies or fragment thereof as well as antigens or specific parts thereof according to this preferred aspect of the invention.

More preferably, the invention provides hybridomas derived from scN2A cells. The invention also provides the monoclonal antibodies, antibodies or fragment thereof as well as antigens or specific parts thereof as well as antigens or specific parts thereof according to this most preferred aspect of the invention.

Even more preferably, the invention provides hybridomas derived from N2A cells. The invention also provides the monoclonal antibodies, antibodies or fragment thereof as well as antigens or specific parts thereof according to this even more preferred aspect of the invention.

More preferably, the invention provides hybridomas that allow selection of antibodies able to prevent conversion of PrP^{C} into PrP^{Sc}. The invention also provides the monoclonal antibodies, antibodies or fragment thereof as well as antigens or specific parts thereof according to this more preferred aspect of the invention.

More preferably, the invention provides hybridomas that allow selection of antibodies able to favour conversion of PrP^{C} into PrP^{Sc}. The invention also provides the monoclonal antibodies, antibodies or fragment thereof as well as antigens or specific parts thereof according to this more preferred aspect of the invention.

Even more preferably, the invention provides hybridoma clones designated #5, #51, #57, #197 and #245 that allow selection of antibodies able to prevent conversion of PrP^{C} into PrP^{Sc} (see example 2). The hybridoma clones are deposited at the European Collection of Cell Cultures (ECACC, http://www.ecacc.org.uk/). The hybridoma clone designated #51 is deposited at the ECACC under Provisional Accession No. 05021601. The hybridoma clone designated #57 is deposited at the ECACC under Provisional Accession No. 05030901. The hybridoma clone designated #245 is deposited at the ECACC under Provisional Accession No. 05021603. The invention also provides the monoclonal antibodies, antibodies or fragment thereof as well as antigens or specific parts thereof according to this even more preferred aspect of the invention. The invention thus provides the monoclonal antibodies generated by hybridoma clone designated #51 deposited at the ECACC under No. 05021601, the monoclonal antibodies generated by hybridoma clone designated #57 deposited at the ECACC under No. 05030901, the monoclonal antibodies generated by hybridoma clone designated #245 deposited at the ECACC under No. 05021603. The antigens identified are either conversion factors (one of the factors implicated in prion replication, e.g. as ApoB identified in EP03101795.7), in their ability to favour conversion of PrP^{C} into PrP^{Sc}, or inhibitors of prion replication, in their ability to prevent conversion of PrP^{C} into PrP^{Sc}. The selected antibodies are either agonistic antibodies towards negative acting factors (i.e. inhibitors of prion replication) or antagonistic antibodies towards positive acting factors (i.e. conversion factors).

Preferably, the invention provides hybridoma clones designated #262, #499 and #608 that allows selection of antibodies able to favour conversion of PrP^{C} into PrP^{Sc} (see example 2). The invention also provides the monoclonal antibodies, antibodies or fragment thereof as well as antigens or specific parts thereof according to this even more preferred aspect of the invention. The antigens identified here are also either conversion factors (one of the factors implicated in prion replication, e.g. as ApoB identified in EP03101795.7), in their ability to favour conversion of PrP^{C} into PrP^{Sc}, or inhibitors of prion replication, in their ability to prevent conversion of PrP^{C} into PrP^{Sc}. But here, the selected antibodies are either agonistic antibodies of conversion factors or antagonistic antibodies of inhibitors of prion replication.

In a sixth aspect, the antibodies of the invention are further capable of regulating a biochemical activity of the antigen according to the fifth aspect of the invention.

In a seventh aspect, the antibodies of the invention are further capable of specifically detecting the antigen according to the fifth aspect of the invention.

Preferably, said antigen is detected by Western blot analysis, ELISA, or immunoprecipitation.

In an eight aspect, the invention provides a pharmaceutical composition comprising a pharmaceutically acceptable carrier and, as an active ingredient, being capable of specifically binding an antibody according to the fourth aspect of the invention or an antigen according to the fifth aspect of the invention.

In an ninth aspect, the invention provides a pharmaceutical composition according to the eight aspect of the invention, wherein said antibody is further capable of regulating a biochemical activity of an antigen according to the fifth aspect of the invention.

In a tenth aspect, the invention provides a composition-of-matter comprising a substrate covalently attached to an antigen according to the fifth aspect of the invention for selectively capturing the antibody capable of specifically binding said antigen.

Preferably, the substrate is an affinity chromatography matrix or selected from the group consisting of a bead, a resin, or a plastic surface and comprises a carbohydrate or a derivative of said carbohydrate.

More preferably, said carbohydrate is selected from the group consisting of agarose, sepharose, and cellulose.

In an eleventh aspect, the invention relates to a method of treatment of a disease caused or aggravated by the activity of an antigen according to the fifth aspect of the invention (the antigen being preferably a conversion factor) comprising the administration of an antibody specifically binding said antigen and being capable of preventing the conversion of PrP^{C} into PrP^{Sc} according to the fourth aspect of the invention.

In a twelfth aspect, the invention relates to a method of treatment of a disease comprising the administration of an antigen according to the fifth aspect of the invention capable of preventing the conversion of PrP^{C} into PrP^{Sc}.

In a thirteenth aspect, the invention relates to the use of an antigen according to the fifth aspect of the invention (the antigen being preferably an inhibitor of prion replication) being capable of preventing the conversion of PrP^{C} into PrP^{Sc} in the manufacture of a medicament for the treatment of a disease.

In a fourteenth aspect, the invention relates to the use of an antibody according to the fourth aspect of the invention being capable of specifically binding the antigen according to the fifth aspect of the invention in the manufacture of a medicament for the treatment of a disease caused or aggravated by the activity of said antigen.

In a fifteenth aspect, the invention provides a device, comprising: a support surface; and an antibody according to the fourth aspect of the invention bound to the surface of the support, the antibody being characterized by an ability to modulate (e.g. prevent or favour) the conversion of PrP^{C} into PrP^{Sc}. A plurality of different antibodies or fragments thereof can be bound to the support surface.

Preferably, the device according to the fifteenth aspect, wherein the antibody or fragment thereof specifically binds to an antigen or a specific portion thereof of a mammal selected from the group consisting of a human, a cow, a sheep, a horse, a pig, a dog, a chicken, a mouse, a rat and a cat.

In a sixteenth aspect, the invention provides the antibody according to the fourth aspect of the invention, further characterized by the ability of said antibody to neutralize PrP^{Sc} infectivity (thus, to prevent conversion of PrP^{C} into PrP^{Sc}).

In a seventeenth aspect, the invention provides the antigen according to the fifth aspect of the invention, further characterized by the ability of said antigen to neutralize PrP^{Sc} infectivity.

In an eighteenth aspect, the invention relates to a method of determining PrP^{Sc} infection in a dead animal, comprising: extracting tissue from an animal that has died; contacting the tissue with an antibody according to the fourth aspect of the invention, wherein the antibody binds to the antigen according to the fifth aspect of the invention specific to the animal that has died; and determining if the antibody has bound to the antigen; wherein presence of the antigen in the tissue is indicative of PrP^{Sc} infection.

In a nineteenth aspect, the invention relates to a method of purifying a material suspected of containing the antigen according to the fifth aspect of the invention, comprising: contacting the material with a sufficient amount of an antibody characterized by its ability to bind the antigen in situ which antibody is bound to a support surface, and removing material not bound to the antibody.

The methods according to the eighteenth or nineteenth aspect, wherein the antibody or fragment thereof specifically binds to an antigen or a specific portion thereof of a mammal selected from the group consisting of a human, a cow, a sheep, a horse, a pig, a dog, a chicken, a mouse, a rat and a cat.

In a twentieth aspect, the invention relates to the use of the antigen according to the fifth aspect of the invention or the antibody according to the fourth aspect of the invention in an assay (e.g. preferably Protein Misfolding Cyclic Amplification (PMCA) assay) for the detection of the formation of PrP^{sc} in a sample.

More preferably, the PMCA assay uses normal brain homogenate as a source of normal PrP^{C} and substrate.

Even more preferably, the PMCA assay uses lipid rafts from infection sensitive neuroblasma cell line N2a as a source of normal PrP^{C} and substrate. Still even more preferably, this cell line N2a is designated #60.

In a twentyfirst aspect, the invention relates to the use of the antigen according to the fifth aspect of the invention or the antibody according to the fourth aspect of the invention in a screening assay for identifying compounds that modulate the conversion of PrP^{c} into PrP^{sc}.

Preferably, the antibody is able to prevent or favour conversion of PrP^{c} into PrP^{sc}.

In a twentysecond aspect, the invention relates to the use of a modulator (e.g. the antibody according to the fourth aspect of the invention) of the antigen according to the fifth aspect of the invention for the preparation of a pharmaceutical preparation for the treatment of a prion disease.

In a twentythird aspect, the invention relates to the use of the antibody according to the fourth aspect of the invention for the preparation of a pharmaceutical formulation for the treatment of a conformational disease. Preferably, said antibody is able to prevent conversion of PrP^{c} into PrP^{sc}.

In a twentyfourth aspect, the invention relates to the use of the antigen according to the fifth aspect of the invention for the preparation of a pharmaceutical formulation for the treatment of a prion disease. Preferably, said antigen is an inhibitor of prion replication.

In a twentyfifth aspect, the invention relates to a method for the diagnosis or detection of a prion disease within a subject suspected of suffering from such a disease which comprises (i) obtaining a sample from the subject; (ii) contacting a sample from said subject with the antigen according to the fifth aspect of the invention or with the antibody according to the fourth aspect of the invention being able to favour conversion of PrP^{c} into PrP^{sc}; (iii) contacting the mixture obtained in step (ii) with PrP^{C} or PrP^{C} containing mixtures; and (iv) determining the presence and/or amount of PrP^{Sc} in said sample.

In a twentysixth aspect, the invention relates to a method for the diagnosis or detection of a prion disease within a subject suspected of suffering from such a disease which comprises (i) obtaining a sample from the subject; (ii) contacting a sample from said subject with the antigen according to the fifth aspect of the invention or with the antibody according to the fourth aspect of the invention being able to favour conversion of PrP^{c} into PrP^{sc} and at least another conversion factor (e.g. Apolipoprotein B or a fragment thereof); (iii) contacting the mixture obtained in step (ii) with PrP^{C} or PrP^{C} containing mixtures; and (iv) determining the presence and/or amount of PrP^{Sc} in said sample.

In a twentyseventh aspect, the invention provides a method of determining a marker that predisposes a subject to a prion disease, comprising (i) obtaining a sample from the subject; (ii) measuring a level of said antibody according to the fourth aspect of the invention or said antigen according to the fifth aspect of the invention; and (iii) correlating said level of protein obtained in said measuring step with the occurrence of a prion disease.

In a twentyeight aspect, the invention provides a method for the detection of PrP^{Sc} within a sample, which assay comprises (i) contacting said sample with said antibody according to the fourth aspect of the invention or with said antigen according to the fifth aspect of the invention; (ii) contacting sample obtained in (i) with PrP^{C} or PrP^{C} containing mixtures; and (iii) determining the presence and/or amount of PrP^{Sc} in said sample.

In a twentyninth aspect, the invention provides a method for the detection of PrP^{Sc} within a sample, which assay comprises (i) contacting said sample with said antibody according to the fourth aspect of the invention or with said antigen according to the fifth aspect of the invention and at least another conversion factor (e.g. Apolipoprotein B or a fragment thereof); (ii) contacting sample obtained in (i) with PrP^{C} or PrP^{C} containing mixtures; and (iii) determining the presence and/or amount of PrP^{Sc} in said sample.

In a thirtieth aspect, the invention provides a method for identifying a compound which modulates the transition of PrP^{C} into PrP^{Sc} comprising: (i) contacting said sample with the antigen according to the fifth aspect of the invent ion or with the antibody according the fourth aspect of the invention (a) in the presence of said modulatory compound and (b) in the absence of said compound; (ii) contacting the mixtures obtained in step (i) a and (i) b with PrP^{C} or PrP^{C} containing mixtures; and (iii) determining the amount of PrP^{Sc} (a) in the presence of said modulatory compound and (b) in the absence of said modulatory compound.

In a thirtyfirst aspect, the invention provides a method for identifying a compound which modulates the transition of PrP^{C} into PrP^{Sc} comprising: (i) contacting said sample with the antigen according to the fifth aspect of the invention or with the antibody according the fourth aspect of the invention and at least another conversion factor (e.g. Apolipoprotein B or a fragment thereof) (a) in the presence of said modulatory compound and (b) in the absence of said compound; (ii) contacting the mixtures obtained in step (i) a and (i) b with PrP^{C} or PrP^{C} containing mixtures; and (iii) determining the amount of PrP^{Sc} (a) in the presence of said modulatory compound and (b) in the absence of said modulatory compound.

In a thirtysecond aspect, the invention provides an assay for the detection of PrP^{Sc} in a sample within a sample, which assay comprises (i) contacting said sample with the antigen according to the fifth aspect of the invention or with the antibody according the fourth aspect of the invention; (ii) contacting the mixture obtained in step (i) with PrP^{C} or PrP^{C} containing mixtures; (iii) determining the presence and/or amount of PrP^{Sc} in said sample.

In a thirtythird aspect, the invention provides an assay for the detection of PrP^{Sc} in a sample within a sample, which assay comprises (i) contacting said sample with the antigen according to the fifth aspect of the invention, or with the antibody according the fourth aspect of the invention and at least another conversion factor and at least another conversion factor (e.g. Apolipoprotein B or a fragment thereof); (ii) contacting the mixture obtained in step (i) with PrP^{C} or PrP^{C} containing mixtures; (iii) determining the presence and/or amount of PrP^{Sc} in said sample.

In a thirtyfourth aspect, the invention provides a screening assay for identifying a compound which modulates the transition of PrP^{C} into PrP^{Sc} comprising: (i) contacting said sample with the antigen according to the fifth aspect of the invention or with the antibody according the fourth aspect of the invention (a) in the presence of said modulatory compound and (b) in the absence of said modulatory compound; (ii) contacting the mixtures obtained in step (i) a and (i) b with PrP^{C} or PrP^{C} containing mixtures; and (iii) determining the amount of PrP^{Sc} (a) in the presence of said compound and (b) in the absence of said modulatory compound.

In a thirtyfifth aspect, the invention provides a screening assay for identifying a compound which modulates the transition of PrP^{C} into PrP^{Sc} comprising: (i) contacting the antigen according to the fifth aspect of the invention, or with the antibody according the fourth aspect of the invention and at least another conversion factor (e.g. Apolipoprotein B or a fragment thereof) (a) in the presence of said modulatory compound and (b) in the absence of said modulatory compound; (ii) contacting the mixtures obtained in step (i) a and (i) b with PrP^{C} or PrP^{C} containing mixtures; and (iii) determining the amount of PrP^{Sc} (a) in the presence of said compound and (b) in the absence of said modulatory compound.

In a thirtysixth aspect, the invention provides a diagnostic kit for use in the assay according to any of the thirtyfifth, thirtysecond, thirtythird or thirthyfourth aspect of the invention, comprising a probe for receiving a sample and the antigen according to the fifth aspect of the invention or with the antibody according the fourth aspect of the invention.

In a thirtyseventh aspect, the invention provides a diagnostic kit for use in the assay according to any of the thirtyfifth, thirtysecond, thirtythird or thirthyfourth aspect of the invention, comprising a probe for receiving a sample and the antigen according to the fifth aspect of the invention or with the antibody according the fourth aspect of the invention and at least another conversion factor (e.g. Apolipoprotein B or a fragment thereof).

The sample can be a biological preparation for which the presence of prion is to be detected for quality control reasons and/or a sample extracted from a subject that is suspected of suffering of such a disease, including a biological extract from a mammal such as cell sample, genetic material, body fluid, including blood, serum, plasma, brain homogenate, cells and lipid rafts.

The kit of the invention comprises kits having multi-well microtitre plate and/or multi-well sonicator.

In a still further embodiment of the invention, is provided an apparatus for use in the methods of the invention or in the assays of the invention. The apparatus of the invention comprises apparatus that have a microtitre plate and/or multi-well sonicator.

In a thirtyeight aspect, the invention provides an apparatus for use in the method of any of the preceding aspects or the assay of any of the preceding aspects.

The invention also provides the antibody, monoclonal antibody, chimeric antibody, fully humanized antibody, anti-anti-ID antibody or fragment thereof being capable of specifically binding said antigen according to the preceding aspects.

Preferably, the antibody is an IgG antibody.

Preferably, the antibody fragment is selected from the group consisting of a single-chain Fv, an Fab, an Fab′, an F(ab′)₂ and a CDR.

Preferably, according to any of the preceding aspects, the antibody or fragment thereof is derived from a human, a cow, a sheep, a horse, a pig, a dog, a chicken, a mouse, a rat and a cat.

Preferably, the disease refers to conformational diseases.

More preferably, the disease is selected from prion disease and from the conformational disease group comprising Alzheimer's Disease, amyotrophic lateral sclerosis (ALS), Pick's disease, Parkinson's disease, Frontotemporal dementia, Diabetes Type II, Multiple myeloma, Plasma cell dyscrasias, Familial amyloidotic polyneuropathy, Medullary carcinoma of thyroid, Chronic renal failure, Congestive heart failure, Senile cardiac and systemic amyloidosis, Chronic inflammation, Atherosclerosis, Familial amyloidosis Gelsolin and Huntington's disease, cerebral amyloid angiopathy (CAA).

Even more preferably, the prion disease is selected from PrP scrapie, FFI (Fatal Familial Insomnia); GSS (Gerstmann-Strassler-Scheinker Disease).

Still even more preferably, the prion disease according to any of the preceding aspects refers to bovine spongiform encephalopathy (BSE) or Creutzfeld-Jacob Disease (CJD). In a preferred embodiment, the prion disease is sporadic, variant, familial or iatrogenic Creutzfeld-Jacob Disease (CJD).

### Antibodies

An IgG antibody preparation of the present invention may be advantageously purified from an anti-serum of the present invention using protein-G affinity purification, is preferably via protein-G immunoprecipitation. An anti-serum derived from an animal immunized, can be used for detecting with optimal sensitivity, via Western immunoblotting analysis, immunoprecipitation and ELISA, the lipid raft antigens.

In general, for applications benefiting from optimal reproducibility, standardization, or precision, a purified antibody or antibody fragment of the present invention capable of specifically binding the target antigen will generally be optimal relative to an unpurified preparation of the present invention.

Purifying the antibody or antibody fragment capable of specifically binding the target antigen can be achieved, for example, by purifying a preparation of the present invention, such as an unpurified anti-serum of the present invention, via affinity chromatography using a substrate covalently attached to the target antigen. Such a substrate-attached target antigen can be used, according to standard affinity chromatography methodology, for selectively capturing the antibody or antibody fragment capable of specifically binding the target antigen.

The substrate is preferably an affinity chromatography matrix. An affinity chromatography matrix, being a substrate optimized for performing affinity chromatography, may be advantageously employed for achieving optimal affinity purification.

Substrates having various structural and chemical characteristics may be employed for performing the purification.

Preferably, the substrate comprises a carbohydrate or a derivative thereof. Preferably, the carbohydrate is agarose, sepharose, or cellulose.

Preferably, the substrate is a bead, a resin, or a plastic surface.

Substrates such as beads, resins, or plastic surfaces comprising carbohydrates such as agarose, sepharose or cellulose are routinely used for practicing affinity chromatography in the art.

Ample guidance for practicing affinity chromatography, such as that employing such substrates, is provided in the literature of the art (for example, refer to: Wilchek M. and Chaiken I., 2000. Methods Mol Biol. 147:1-6; Jack G W. Immunoaffinity chromatography. Mol Biotechnol 1, 59-86; Narayanan S R., 1994. Journal of Chromatography A 658:237-258; Nisnevitch M. and Firer M A., 2001. J Biochem Biophys Methods 49:467-80; Janson J C. & Kristiansen T. in: “Packings and Stationary Phases in Chromatography Techniques” (ed. Unger, K K.) pp. 747 (Marcel Dekker, New York, 1990); Clonis, Y. D. in: “HPLC of Macromolecules: A Practical Approach”, pp. 157 (IRL Press, Oxford, 1989); Nilsson J. et al., 1997. Protein Expr Purif. 11:1-16).

Alternatively, a preparation of the present invention can be purified using a variety of standard protein purification techniques, such as, but not limited to, ion exchange chromatography, filtration, electrophoresis, hydrophobic interaction chromatography, gel filtration chromatography, reverse phase chromatography, concanavalin A chromatography, chromatofocusing and differential solubilization.

Purifying the antibody or antibody fragment capable of binding the target antigen with a desired affinity from a preparation of the present invention, such as an unpurified anti-serum of the present invention, can be achieved, for example, via affinity chromatography purification of an unpurified—or more preferably a protein-G purified—anti-serum of the present invention, by using the target antigen as an affinity ligand, and via selective elution of a substrate-bound antibody or antibody fragment under conditions of controlled stringency (for example under conditions of controlled pH and/or salt concentration). In particular, an antibody or antibody fragment of the present invention capable of binding the target antigen with a maximal affinity may be conveniently obtained by elution under conditions of effectively maximal stringency (for example under conditions of effectively maximal or minimal pH and/or maximal salt concentration). Typically, an antibody or antibody fragment may be bound to a substrate-attached cognate antigen thereof under conditions of physiological pH and salt concentration, and such an antibody or antibody fragment may typically be eluted from the substrate by decreasing the pH to 2.5 or lower, or by increasing the pH to 11 or higher.

It will be appreciated by the ordinarily skilled artisan that an antibody or antibody fragment having an affinity characterized by a dissociation constant of up to 10⁻¹² for a cognate antigen can be obtained using common art techniques.

As described hereinabove, the preparation may advantageously comprise an antibody or antibody fragment attached to any of various types of detectable molecule.

A preparation of the present invention comprising an antibody or antibody fragment attached to a detectable molecule can be used for detecting the target antigen specifically bound by the antibody or antibody fragment.

The preparation may comprise an antibody or antibody fragment attached to any of numerous types of detectable molecule, depending on the application and purpose.

For example, depending on the application and purpose, the detectable molecule may advantageously be a fluorophore, an enzyme, a light-emitting molecule, or a radioisotope.

Preferably, the detectable molecule is an enzyme or a protein.

An enzyme may be advantageously utilized for enabling detection of the target antigen via any of various enzyme-based detection methods. Examples of such methods include, but are not limited to, enzyme linked immunosorbent assay (ELISA; for example, for detecting the target antigen in a solution), enzyme-linked chemiluminescence assay (for example, for detecting the complex in an electrophoretically separated protein mixture), and enzyme-linked histochemical assay (for example, for detecting the complex in a fixed tissue).

Numerous types of enzymes may be employed for detecting the target antigen, depending on the application and purpose.

Examples of suitable enzymes include, but are not limited to, horseradish peroxidase (HPR), β-galactosidase, and alkaline phosphatase (AP).

Ample guidance for practicing enzyme-based molecular detection methods is provided in the literature of the art (for example, refer to: Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

A preparation of the present invention comprising an antibody or antibody fragment attached to a fluorophore may be advantageously employed for detecting the target antigen via any of numerous fluorescence-based molecular detection methods. Depending on the application and purpose, such methods include, but are not limited to, fluorescence activated flow cytometry (FACS; for example for characterizing expression or display of the target antigen in a suspended cell population), fluorescence confocal microscopy (for example, for detecting the molecule in a dead or living cell or tissue in three dimensions), fluorescence in-situ hybridization (FISH), fluorescence resonance energy transfer (FRET; for example, for detecting a specific intermolecular association involving the target antigen), fluorescence histochemistry (for example, for detecting the molecule in a fixed histological sample), and the like. Various types of fluorophores, depending on the application and purpose, may be employed for detecting the target antigen.

Examples of suitable fluorophores include, but are not limited to, phycoerythrin, fluorescein isothiocyanate (FITC), Cy-chrome, rhodamine, green fluorescent protein (GFP), blue fluorescent protein (BFP), Texas red, and the like.

Ample guidance regarding fluorophore selection, methods of linking fluorophores to various types of molecules, such as an antibody or antibody fragment of the present invention, and methods of using such fluorescent immunoconjugates for detecting molecules is available in the literature of the art [for example, refer to: Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.].

Examples of suitable light-emitting molecules include luminol.

Examples of suitable radioisotopes include [125]iodine, [35]sulfur, [3]hydrogen, [32]phosphorus, etc.

The detectable molecule may be attached to the antibody or antibody fragment in various ways, depending on the application and purpose, and on the nature of the molecules involved. Ample guidance for attaching a detectable molecule to an antibody or antibody fragment is provided in the literature of the art [for example, refer to: “Using Antibodies: A Laboratory Manual”, Ed Harlow, David Lane (eds.), Cold Spring Harbor Laboratory Press (1999); also, refer to the extensive guidelines provided by The American Chemical Society, for example at: http://www.chemistry.org/portal/Chemistry]. One of ordinary skill in the art, such as a chemist, will possess the required expertise for suitably practicing such chemical synthesis techniques.

Accordingly, a preparation of the present invention comprising an antibody or antibody fragment attached to a detectable molecule can be used for efficiently and uniquely detecting the target antigen in essentially any context.

Depending on the application and purpose, the preparation may advantageously be a preparation of any of various types of antibody fragments.

As already mentioned, the antibody fragment is preferably a single-chain Fv (scFv), or more preferably an Fab, Fab′, F(ab′)₂ or CDR.

An antibody fragment has the advantage of being smaller than a parental antibody from which it is derived while retaining substantially identical target-antigen binding specificity, or both binding specificity and binding affinity, as the parental antibody. Thus, an antibody fragment, by virtue of being smaller than the parental antibody, will thereby generally have superior biodistribution, and diffusion properties (for example, systemically in-vivo, or in isolated tissues) than the latter. An antibody fragment substantially lacking an Fc region, such as a single-chain Fv, an Fab′, an Fab an F(ab′)₂ or a CDR, is advantageous for applications involving exposure of the preparation to a molecule capable of specifically binding such an Fc region, and in which such binding is undesirable. Typically this may involve an undesired binding of an Fc region exposed to a cognate Fc receptor, or an Fc-binding complement component (for example, complement component C1q, present in serum). Fc receptors are displayed on the surface of numerous immune cell types, including: professional APCs, such as dendritic cells; B lymphocytes; and granulocytes such as neutrophils, basophils, eosinophils, monocytes, macrophages, and mast cells. Thus, the absence of an Fc region from the antibody fragment may be particularly advantageous for avoiding undesired an Fc receptor-mediated immune cell activation or a complement component-mediated complement cascade, particularly when administering the preparation in-vivo to an individual.

An F(ab′)₂ is a fragment of an antibody molecule containing a divalent antigen-binding portion of an antibody molecule.

An F(ab′)₂ preparation of the present invention may be conveniently obtained using standard art methods by treating an antibody preparation of the present invention, such as an anti-serum of the present invention, with the enzyme pepsin. The resultant F(ab′)₂ product is a 5S particle.

An Fab, or Fab′ is a fragment of an antibody molecule containing a monovalent antigen-binding portion of an antibody.

The CDR can be generated e.g. as described in EP0585939 or as described by Strandberg et al. (Protein Eng. 2001 January; 14(1): 67-74). The CDR according to the invention can be a modified CDR, which has enhanced effect on th e modulation of lipid raft antigen. An example for methods of modification of active peptides is described by Sawa et al. 1999 (J. Med. Chem. 42, 3289-3299).

An Fab′ preparation of the present invention may be conveniently obtained using standard art methods by treating an antibody preparation of the present invention, such as an anti-serum of the present invention, with the enzyme pepsin, followed by reduction of the resultant F(ab′)₂ into. Such reduction may be effected using a thiol reducing agent, and optionally using a blocking group for the sulfhydryl groups resulting from cleavage of disulfide linkages. Such treatment generates two monovalent 3.5S Fab's an Fc fragment.

An Fab preparation may be conveniently obtained using standard art methods by treating an antibody preparation of the present invention, such as an anti-serum of the present invention, with the enzyme papain to yield the intact light chain and a portion of heavy chain composed of the variable and C_{H}1 domains.

Ample guidance for generating an antibody fragment by enzymatic treatment of an antibody is provided in the literature of the art (for example, refer to: Goldenberg, U.S. Pat. Nos. 4,036,945 and 4,331,647; Porter R R., 1959. Biochem J. 73:119-126).

A single chain Fv (also referred to in the art as “scFv”) is a single chain molecule including the variable region of the light chain and the variable region of the heavy chain, linked by a suitable polypeptide linker.

An F(ab′)₂, Fab′, Fab, or single-chain Fv or CDR preparation of the present invention may be obtained using recombinant techniques.

Obtaining a recombinant antibody fragment is effected by isolating mRNA of B lymphocytes of animals immunized with the target antigen, generating cDNA from the mRNA via RT-PCR, and using the CDNA to construct an antibody fragment phage-display library. B lymphocytes can be conveniently isolated from the spleen, or, alternately from the blood, bone-marrow, or lymph nodes of the immunized animal.

It will be appreciated that the above-described methodology can be used to obtain a monoclonal antibody fragment preparation of the present invention having essentially any desired target antigen-binding affinity and/or specificity. Such a preparation can be utilized in various applications benefiting from a reagent capable of binding the target antigen with such defined target antigen-binding characteristics.

Since an Fab′ is essentially similar in structure to an Fab, a preparation of the present invention comprising an Fab′ may be employed essentially interchangeably with one comprising an Fab, where such Fab′ and Fab comprise essentially the same heavy and light chain variable regions. For applications, as will usually be the case, benefiting from a preparation of the present invention comprising an antibody fragment capable of binding the target antigen with maximal affinity, an F(ab′)₂ preparation of the present invention may superior to an Fab, Fab′ or scFv preparation of the present invention, due to the divalent binding of an F(ab′)₂ to the target antigen relative to the monovalent binding of such a monovalent antibody fragment.

As mentioned hereinabove, depending on the application and purpose, the antibody or antibody fragment preparation may originate from any of various mammalian species

An antibody or antibody fragment preparation of the present invention originating from a desired species may be derived from serum of the animal of such species immunized with the target antigen.

A preparation of the present invention of a human or humanized antibody or antibody fragment may be preferable for applications involving administration of the preparation to an individual. For example, a human or humanized antibody or antibody fragment will generally tend to be optimally tolerated immunologically, and hence will display an optimal half-life in-vivo in a human, and will thereby display optimal effectiveness. Further guidance regarding production and exploitation of human or humanized antibodies is provided hereinbelow.

The preparation may be used per se or it can be formulated as an active ingredient in a pharmaceutical composition.

Thus, according to the present invention there is provided a pharmaceutical composition comprising a pharmaceutically acceptable carrier and, as an active ingredient, the antibody or antibody fragment of the present invention.

Methods of formulating the antibody or antibody fragment of the present invention as an active ingredient in a pharmaceutical composition, and methods of exploiting such a pharmaceutical composition are described hereinbelow.

Preferably, administering the antibody or antibody fragment is effected by administering the pharmaceutical composition of the present invention comprising the antibody or antibody fragment of the present invention as an active ingredient.

The antibody or antibody fragment is preferably administered so as to achieve a sufficient level of antibody fragment bound to the target antigen so as to achieve a desired regulation of the biochemical activity.

An ordinarily skilled artisan, such as a physician, more preferably a physician specialized in the disease, will possess the required expertise for determining a suitable therapeutic protocol, including a suitable route of administration, and a suitable dosage of the antibody or antibody fragment for effectively treating the disease according to the teachings of the present invention.

As described hereinabove, the target antigen, which is a polypeptide, may be obtained in various ways.

Preferably, the target antigen is obtained via standard chemical synthesis methodology.

The target antigen may be chemically synthesized using, for example, standard solid phase techniques. Such techniques include exclusive solid phase synthesis, partial solid phase synthesis methods, fragment condensation, classical solution synthesis. Solid phase polypeptide synthesis procedures are well known in the art [for example, refer to Stewart et al., in “Solid Phase Peptide Synthesis”, 2nd ed., Pierce Chemical Company, (1984)].

A synthetic polypeptide can be purified by preparative high performance liquid chromatography procedure, such as described by Creighton T. [Proteins, structures and molecular principles, W. H. Freeman and Co. N.Y. (1983)] and its amino acid sequence may be confirmed via standard amino acid sequencing procedures.

As described hereinabove, the preparation is preferably derived by immunizing a mammal with the target antigen.

Generating the preparation in-vivo may be advantageously effected by repeated injection of the target antigen into a mammal in the presence of adjuvant according to a schedule which boosts production of antibodies in the serum. In cases wherein the target antigen is too small to elicit an adequate immunogenic response (referred to as a “hapten” in the art), the hapten can be coupled to an antigenically neutral carrier such as keyhole limpet hemocyanin (KLH) or serum albumin [e.g., bovine serum albumin (BSA)] carriers (for example, refer to U.S. Pat. Nos. 5,189,178 and 5,239,078). Coupling a hapten to a carrier can be effected using various methods well known in the art. For example, direct coupling to amino groups can be effected and optionally followed by reduction of the imino linkage formed. Alternatively, the carrier can be coupled using condensing agents such as dicyclohexyl carbodiimide or other carbodiimide dehydrating agents. Linker compounds can also be used to effect the coupling; both homobifunctional and heterobifunctional linkers are available from Pierce Chemical Company, Rockford, Ill. The resulting immunogenic complex can then be injected into suitable mammalian subjects such as cows, sheeps, mice, rabbits, and the like. Following in-vivo generation of an antibody, its serum titer in the host mammal can readily be measured using immunoassay procedures which are well known in the art.

As described hereinabove, the preparation may advantageously comprise a humanized antibody or antibody fragment.

Humanized antibodies or antibody fragments are genetically engineered chimeric antibodies or antibody fragments having—preferably minimal—portions derived from non human antibodies. Humanized antibodies include antibodies in which complementary determining regions of a human antibody (recipient antibody) are replaced by residues from a complementarity determining region of a non human species (donor antibody) such as mouse, rat or rabbit having the desired functionality. In some instances, Fv framework residues of the human antibody are replaced by corresponding non human residues. Humanized antibodies may also comprise residues which are found neither in the recipient antibody nor in the imported complementarity determining region or framework sequences. In general, the humanized antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanized antibodies optimally also include at least a portion of an antibody constant region, such as an Fc region, typically derived from a human antibody (see, for example, Jones et al., 1986. Nature 321:522-525; Riechmann et al., 1988. Nature 332:323-329; and Presta, 1992. Curr. Op. Struct. Biol. 2:593-596). Methods for humanizing non human antibodies or antibody fragments are well known in the art. Generally, a humanized antibody has one or more amino acid residues introduced into it from a source which is non human. These non human amino acid residues are often referred to as imported residues which are typically taken from an imported variable domain. Humanization can be essentially performed as described (see, for example: Jones et al., 1986. Nature 321:522-525; Riechmann et al., 1988. Nature 332:323-327; Verhoeyen et al., 1988. Science 239:1534-1536; U.S. Pat. No. 4,816,567) by substituting human complementarity determining regions with corresponding rodent complementarity determining regions. Accordingly, such humanized antibodies are chimeric antibodies, wherein substantially less than an intact human variable domain has been substituted by the corresponding sequence from a non human species. In practice, humanized antibodies may be typically human antibodies in which some complementarity determining region residues and possibly some framework residues are substituted by residues from analogous sites in rodent antibodies. Human antibodies or antibody fragments can also be produced using various techniques known in the art, including phage display libraries [see, for example, Hoogenboom and Winter, 1991. J. Mol. Biol. 227:381; Marks et al., 1991. J. Mol. Biol. 222:581; Cole et al., “Monoclonal Antibodies and Cancer Therapy”, Alan R. Liss, pp. 77 (1985); Boemer et al., 1991. J. Immunol. 147:86-95). Humanized antibodies can also be made by introducing sequences encoding human immunoglobulin loci into transgenic animals, e.g., into mice in which the endogenous immunoglobulin genes have been partially or completely inactivated. Upon antigenic challenge, human antibody production is observed in such animals which closely resembles that seen in humans in all respects, including gene rearrangement, chain assembly, and antibody repertoire. Ample guidance for practicing such an approach is provided in the literature of the art (for example, refer to: U.S. Pat. Nos. 5,545,807, 5,545,806, 5,569,825, 5,625,126, 5,633,425, and 5,661,016; Marks et al., 1992. Bio/Technology 10:779-783; Lonberg et al., 1994. Nature 368:856-859; Morrison, 1994. Nature 368:812-13; Fishwild et al., 1996. Nature Biotechnology 14:845-51; Neuberger, 1996. Nature Biotechnology 14:826; Lonberg and Huszar, 1995. Intern. Rev. Immunol. 13:65-93).

### Formulations, Administration and Dosage

Techniques for formulation and administration of drugs may be found in “Remington's Pharmaceutical Sciences,” Mack Publishing Co., Easton, Pa., latest edition, which is incorporated herein by reference.

The above-mentioned modulatory compounds, antigens or specific portion thereof, or antibodies or fragment thereof of the present invention may be administered by any means that achieves the intended purpose. For example, administration may be by a number of different routes including, but not limited to subcutaneous, intravenous, intradermal, intramuscular, intraperitoneal, intra-cerebral, intrathecal, intranasal, oral, rectal, transdermal, intranasal or buccal. Preferably the compounds of the invention are administered by subcutaneous, intramuscular or intravenous injection or infusion. Suitable routes of administration of the pharmaceutical composition may, for example, include oral, rectal, transmucosal, especially transnasal, intestinal or parenteral delivery, including intramuscular, subcutaneous and intramedullary injection as well as intrathecal, direct intraventricular, intravenous, intraperitoneal, intranasal, or intraocular injection.

A variety of aqueous carriers can be used, e.g., water, buffered water, 0.4% saline, 0.3% glycine and the like. These solutions are sterile and generally free of particulate matter. The compositions may contain pharmaceutically acceptable auxiliary substances as required to approximate physiological conditions such as pH adjusting and buffering agents, toxicity adjusting agents and the like, for example sodium acetate, sodium chloride, potassium chloride, calcium chloride, sodium lactate, histidine and arginine. The concentration of the antibodies in these formulations can vary widely, i.e., from less than about 0.01%, usually at least about 0.1% to as much as 5% by weight and are selected primarily based on fluid volumes, and solubilities in accordance with the particular mode of administration selected.

Thus, a typical pharmaceutical composition for injection could be made up to contain 1 ml sterile buffered water, and 1-100 mg of an antibody. A typical composition for intravenous infusion can be made up to contain 250 ml of sterile Ringer's solution, and 10 mg of the inhibitor. Actual methods for preparing parentally administerable compositions are known or apparent to those skilled in the art and are described in more detail in, for example, Remington's Pharmaceutical Science (15th Ed., Mack Publishing Company, Easton, Pa., 1980), which is incorporated herein by reference.

The antibodies of this invention can be frozen or lyophilized for storage and reconstituted in a suitable carrier prior to use depending on the physical characteristics of the inhibitors. This technique has been shown to be effective with conventional antibodies and art-known lyophilization and reconstitution techniques can be employed. For the purpose of treatment of disease, the appropriate dosage of antibodies will depend on the severity and course of disease, the patient's clinical history and response, the toxicity of the inhibitors, and the discretion of the attending physician. The inhibitors are suitably administered to the patient at one time or over a series of treatments. The initial candidate dosage may be administered to a patient. The proper dosage and treatment regime can be established by monitoring the progress of therapy using conventional techniques known to the people skilled of the art.

The amount of active ingredients that may be combined with the carrier materials to produce a single dosage form will vary depending upon the host treated and the particular mode of administration. It will be understood, however, that the specific dose level for any particular patient will depend upon a variety of factors, including the activity of the specific inhibitor employed, the age, body weight, general health, sex, diet, time of administration, route of administration, and rate of excretion, drug combination and the severity of the particular disease undergoing therapy, and can be determined by those skilled in the art.

Parenteral administration can be by bolus injection or by gradual perfusion over time. A typical regimen for preventing, suppressing, or treating prion related disorders, comprises either (1) administration of an effective amount in one or two doses of a high concentration of modulatory in the range of 0.5 to 10 mg of peptide, more preferably 0.5 to 10 mg of peptide, or (2) administration of an effective amount of the peptide in multiple doses of lower concentrations of modulatory compounds in the range of 10-1000 μg, more preferably 50-500 μg over a period of time up to and including several months to several years. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

Preparations for parenteral administration include sterile aqueous or non-aqueous solutions, suspensions, and emulsions, which may contain auxiliary agents or excipients which are known in the art. Suitable formulations for parenteral administration include aqueous solutions of the active compounds in water-soluble form, for example, water-soluble salts. In addition, suspension of the active compound as appropriate oily injections suspensions may be administered.

Alternately, one may administer the pharmaceutical composition in a local rather than systemic manner, for example, via injection of the pharmaceutical composition directly into a tissue region of the individual.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the active ingredients and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa buffer or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (antibody or antibody fragment of the present invention) capable of preventing, alleviating or ameliorating symptoms of the disease, or prolong the survival of the individual being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in-vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in-vitro, in cell cultures or experimental animals. The data obtained from these in-vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (for example, refer to Fingl, et al., 1975, in “The Pharmacological Basis of Therapeutics”, Ch. 1 p. 1).

Dosage amount and interval may be adjusted individually to provide plasma or brain levels of the active ingredients sufficient to exert a desired therapeutic effect (minimal effective concentration, MEC). The MEC will vary for each preparation, but can be estimated from in-vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the individual being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising an antibody or antibody fragment of the invention formulated in a compatible pharmaceutical carrier may also be prepared, placed in an appropriate container, and labeled for treatment of an indicated condition, as if further detailed above.

It is expected that during the life of this patent many relevant medical diagnostic techniques will be developed and the scope of the term “detecting” when relating to the target antigen is intended to include all such new technologies a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Before the present antibodies, assays and methods for producing an using such are disclosed and described, it is to be understood that this invention is not limited to particular antibodies, assays or method as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

The invention will now be described by means of the following Examples, which should not be construed as in any way limiting the present invention. The Examples will refer to the Figures specified here below.

### ABBREVIATIONS

Apo B (Apolipoprotein B; Apo E (apolipoprotein E); Apo J (Apolipoprotein J); BCA (Bicinchoninic Acid); CHAPS (3-((3-cholamidopropyl)dimethylammonio)-1-propanesulfonate); CNS (central nervous system); BSE (bovine spongiform encephalopathy); CJD (Creutzfeldt-Jakob Disease); DIM (Detergent-insoluble Membrane); DRM (Detergent-Resistant Membrane); DTT (1,4-Dithio-D,L-threitol); IPG (Immobilized PH Gradient); IEF (Isoelectric Focusing); FFI (Fatal Familial Insomnia); GSS (Gerstmann-Strassler-Scheinker Disease); hr (hour); HRP (Horseradish Peroxidase); kDa (KiloDalton); LDL (Low Density Lipoprotein); g (microgram); l (microliter); min (minute); MβCD (methyl-β-cyclodextrin); mM (millimolar); MS (mass spectrometry); PBS (Phosphate Buffered Sulfate); PK (proteinase K); PMCA (Protein Misfolding Cyclic Amplification); PMSF (Phenylmethanesulfonyl Fluoride); PrP (prion protein); PrP^{C} (normal, non-pathogenic conformer of PrP); PrP^{Sc} (pathogenic or “scrapie” isoform of PrP which is also the marker for prion diseases); PVDF (polyvinylidene difluoride); RPM (Rotation per minute); RML (Rocky Mountain Laboratory); RT-PCR (reverse transcriptase polymerase chain reaction); SDS (Sodium Dodecyl Sulfate); V (Volt); Vol. (volume), AD for Alzheimer's disease; CAA for cerebral amyloid angiopathy; Hu for human; HuPrP for human prion protein; Mo for mouse; MoPrP for mouse prion protein; SHa for a Syrian hamster; SHaPrP for a Syrian hamster prion protein; PAMAM for polyamidoamide dendrimers; PEI for polyethyleneimine; PK for proteinase K PPI for polypropyleneimine; PrP^{Sc} for the scrapie isoform of the prion protein; PrP for the cellular contained common, normal isoform of the prion protein; PrP 27-30 or PrP^{Sc} 27-30 for the treatment or protease resistant form of PrP^{Sc}; MoPrP^{Sc} for the scrapie isoform of the mouse prion protein; N2a for an established neuroblastoma cell line used in the present studies; ScN2a for a chronically scrapie-infected neuroblastoma cell line; ALS for amyotrophic lateral sclerosis; HD for Huntington's disease; FTD for frontotemporal dementia; SOD for superoxide dismutase.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al., (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al., (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

The invention will be illustrated by means of the following examples which are not to be construed as limiting the scope of the invention. The following examples illustrate preferred compounds and methods for determining their biological activities.

### Example 1

### Introduction

Neuroblastoma cell line derived from mice (N2a) was used in the present invention because it is one of the few cell lines that can be infected with prion (Butler et al. 2001). Two N2a subclones either resistant or sensitive to infection (herein referred to as #23 and #60 respectively) were isolated. These subclones were selected because they displayed similar morphology, growth rates and levels of PrP expression. Furthermore isolation of PrP cDNA from both cell lines revealed identical coding sequences. All these data together suggest that the phenotypic differences between the sensitive and resistant subclones are not due to differences in the expression, localisation or primary sequence of PrPC but rather to the presence or absence of other factors within the lipid rafts involved in the process of conversion.

In order to identify these factors, a “monoclonal antibody approach” was used in which a battery of rat monoclonal antibodies (Mabs) were produced against total lipid rafts pooled from both subclones. A total of 631 Mabs were tested in an intensive primary screening campaign comprising: (i) ELISA for interaction with N2a-derived lipid rafts and (ii) FACS analysis comparing both sensitive and resistant subclones. Thus, 464 Mabs were selected and tested in duplicates for their ability to interfere with a cell-based prion replication assay. Interestingly, 22 out of 464 Mabs significantly inhibited replication without apparently affecting cell growth. Furthermore, none of them turned out to be specific for PrP^{C}, suggesting that Mabs are directed against other components of the lipid rafts.

### Material and Methods

PrP scrapie used as infection inoculum is RML (Rocky Mountain Laboratory) strain.

Anti-PrP 6H4 monoclonal antibodies were purchased from Prionics.

Proteinase K was obtained from Boerhinger Ingelheim and methyl-β-cyclodextrin from Sigma.

Mouse neuroblastoma N2a cell line was obtained from ATCC.

### Cell Culture

N2a mouse neuroblastoma cells (ATCC, CCL-131) were grown in complete medium (DMEM (Gibco), 10% FCS, 100 U/ml penicillin-streptomycin (Gibco), 2 mM L-glutamine (Gibco)). Subclones of the parental cell line were derived from single cells by limit dilution as described previously (EP03101795.7). Briefly, a growing culture was diluted to a density of 5 cells/ml and 100 μl was transferred to individual wells of a 96 well plate and cultured for 1 week. The individual cultures were examined microscopically to determine those wells which contained a single focus of growing cells. The single cell derived cultures were then transferred to 24 well plates and serially passaged every 3-4 days at 1:15 dilution to maintain stocks. A total of 64 cultures were isolated, and all were tested for sensitivity to infection by the RML strain of PrP^{Sc}. To do this, 4 μl of a 10% late stage infected brain extract was added per well of newly passaged cells, and the cultures were left for a further 4 days to reach confluence. Cells were serially passaged thereafter in the absence of PrP^{Sc}. Tests showed that all trace of the initial inoculum disappeared by passage 4. At this and later passages individual cultures were tested for the presence of PrP^{Sc} using a cell culture dot blotting procedure (see below).

### Lipid Rafts Preparation

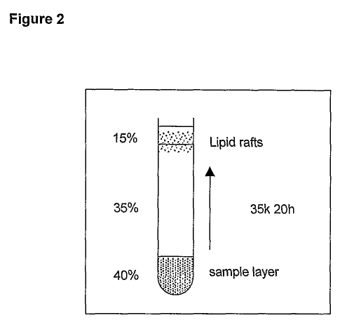

Lipid rafts were purified following the protocol described by Fivaz et al. with minor modifications (Fivaz et al. 2000). Subconfluent cultures of N2a cells in 15 cm culture dishes were washed in PBS and collected by centrifugation 1000 g for 6 min. The cell pellet was resuspended in 1 ml cold raft buffer (1% Triton in PBS, and a cocktail of complete protease inhibitors (Boehringer Mannheim)). Cells were disrupted by seven passages through a 22G needle followed by incubation for 30 min at 4° C. with gentle agitation. 2 volumes 60% (w/v) sucrose in PBS was added and the lysate was transferred to a SW41 centrifuge tube. The lysate was carefully overlaid with 7 ml 35% (w/v) sucrose and 1 ml 15% (w/v) sucrose both in PBS and centrifuged 20 hr at 35,000 RPM (FIG. 2). The lipid rafts were recovered in the top 1 ml of the gradient.

Membranes were concentrated by addition of 10 volumes cold PBS and centrifugation at 100,000 g for 1 hr. Lipid rafts were resuspended in PBS and protein concentration was determined by Bradford coloration (Biorad). Following this protocol, cells from 18×15 cm culture dishes yielded 0.8-1.2 mg of protein.

### Generation of Monoclonal Antibodies

Lipid rafts from sensitive (non-infected) and resistant cells were pooled, concentrated by centrifugation and resuspended in sterile PBS. Aliquots of 150 μl containing 200 μg of proteins were mixed with an equal volume of adjuvant (MPL+TDM Emulsion, RIBI Immunochem Research, Inc. Hamilton, Mont. 59840) and injected subcutaneously into the hind foot of female OFA rats. Similar injections were made 1 week and 4 weeks later. 3 days after the third injection, the lymph node and the spleen were dissected, dispersed in collagenase and DNase dissolved in Iscove's FCS-free Medium (collagenase IV, 2.4 μg/ml, Worthington Biochemical Corp.; DNase, 0.1%, Sigma). Lymph node cells were fused at 37° C. with myeloma cells (SP 2/0) in the presence of PEG1000 (Sigma). Fused cells were distributed in 96-well plates (flat bottom) in order to have 0.8 cell/well and grown in HAT selection medium (DMEM medium (Gibco), 10% FCS, 2.5×10⁻² mM 2-β-mercaptoethanol (Fluka), 1.68 mM L-glutamine (Gibco), 8.39×10⁻² U/ml bovine insulin (Sigma), 0.5 mM sodium pyruvate (Sigma), 1 mM oxalacetic acid (Fluka), 85 U/ml penicillin-streptomycin (Gibco), 8.4×10⁻² mM hypoxanthine (Fluka), 0.84 μM aminopterine (Fluka), 1.34×10⁻² mM thymidine (Fluka)) on a feeder layer of dissected spleen cells at 37° C. in 5% CO₂/95% air. Cell growth was checked every 2 days under the microscope. On day 4 of culture, 100 μl of fresh selection medium was added. Between day 10-12 post-fusion, the hybridomas in some wells had grown enough to start the screening protocol.

### ELISA (Enzyme-Linked Immunosorbent Assay)

96 well plates (NUNC Immunoplate) were coated with 100 μl/well of N2a total lipid rafts (10 μg/ml protein) and were left overnight at 4° C. (coating buffer: 0.015M Na₂CO₃, 0.034M NaHCO₃, pH 9.4 adjusted). Plates were then submitted to four washes with 200 μl/well of PBS-0.05% Tween 20 and blocked 1 hour with 100 μl/well of PBS-10% FCS at room temperature (RT). After two washes, plates were incubated for 2 hours at RT with 50 μl/well of undiluted hybridoma supernatant (primary antibody). After two washes, 100 μl/well of a secondary antibody (mouse anti-IgG rat HRP-conjugated, 200 ng/ml, Jackson ImmunoResearch) were added. Plates were then washed four times and Ab-binding was revealed by adding 100 μl/well of HRP substrate (0.02M Na₂HPO₄, 0.01M citric acid, 0.03% H₂O₂, one 5 mg tablet of o-phenylenediamine for 10 ml of solution, Sigma). The reaction was stopped by addition of 100 μl/well of H₂SO₄ 20% (3.8M). Optical density was read at 490/570 nm with a spectrophotometer (Multiskan EX, Labsystems). A primary non-specific rat monoclonal antibody was used as negative control (rat anti-FDC-M2, 20 μg/ml, made in house) and mouse anti-6H4 (200 ng/ml, Prionics) was used as a positive control. The blank was measured in the absence of primary antibody. Antibody dilutions were done in PBS, 0.05% Tween 20.

### FACS (Fluorescence Activated Cell Sorter) Analysis

Cells were harvested at 1500 RPM/5 min (Heraeus, Megafuge 1.0R), washed in FACS buffer (PBS, 1% BSA, 0.01% Na-azide) and resuspended in FACS buffer at a concentration of 2×10⁶ cells/ml. Then 2×10⁵ cells/well were distributed in 96-well “V”-bottom plates (NUNC). Cells were pelleted at 1500 rpm for 2 min (Heraeus, Megafuge 1.0R) and the supernatant was discarded. Cells were then incubated 30 min at RT with 50 μl of undiluted hybridoma supernatant. After two washes with FACS buffer, cells were incubated for 30 min at RT with 100 μl of secondary fluorescent antibody (goat anti-IgG rat conjugated to R-Phycoerythrin (rPE), 25 μg/ml, Jackson ImmunoResearch). After two washes with FACS buffer, cells were resuspended in 50 μl of FACS buffer and fixed with 50 μl of paraformaldehyde (PFA) 1%. Plates were then analysed in the multiwell autosampler of the FACSCalibur (Becton Dickinson). A primary irrelevant antibody was used as negative control (rat anti-FDC-M2 20 μg/ml, made in house) and mouse anti-6H4 (0.5 μg/ml, Prionics,) was used as a positive control.

### Western Blotting

Lipid rafts derived from either the sensitive or resistant subclones were analysed on SDS NuPage 4-12% Bis-Tris pre-cast gels (Invitrogen). Following electrophoresis, proteins were transferred to PVDF membranes at 100V for 1 hr in a solution containing 192 mM glycine, 25 mM Tris, 20% methanol. Non specific binding was blocked by incubation with 5% milk dissolved in PBS for 1 hr and the membrane was then treated by 1 hr incubations in primary antibody, followed by HRP conjugated secondary antibody each diluted as appropriate in PBS, 0.3% Tween 20. Western blots were developed by ECL (Amersham).

### Cell Culture Dot Blotting

The formation of PrP^{Sc} in N2a cells was monitored using a dot-blotting procedure modified from the protocol described by Bosque and Prusiner (Bosque et al. 2000). Briefly cells growing in 24 well culture dishes were washed with PBS and lysed for 20 minutes in 80 μl lysis buffer (50 mM Tris pH 7.4, 150 mM NacCl, 0.5% Deoxycholate, 0.5% Triton X100) containing 40U DNasel (Sigma D-5025). To monitor the effects of treatments on cell growth, duplicate 2 μl aliquots of the lysate were removed from each well for protein determination. Proteinase K was added to a final concentration of 20 μg/ml and plates were incubated with gentle agitation for 1 h at 37° C. Digestion was stopped by addition of PMSF to a final concentration of 2 mM. For dot blotting, aliquots of the proteinase K-digested cell lysates were spotted onto humid PVDF membranes (Immobilon-P, Millipore). The membrane was transferred immediately to 3M guanidinium thiocyanate for 10 minutes to denature proteins, rinsed 5 times with H₂O and processed as for Western Blotting using mouse anti-6H4 (Prionics) as described above. For quantitative studies the chemiluminescent signal from each spot was determined directly using the Kodak 440 Digital Image Station and normalized for protein content per well.

### Results

### N2a Subclones

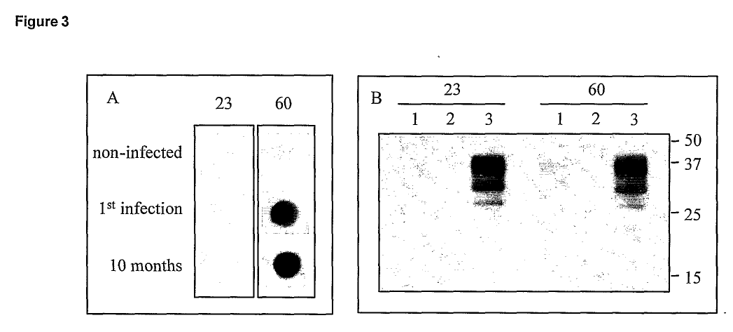

Individual subclones of the N2a parental cell line had been isolated and tested for their sensitivity to infection by the mouse scrapie prion strain RML (in EP03101795.7). Several sensitive and resistant subclones had been selected for further study. The work described in the present claim focused on two of these subclones referred to as #23, a PrPSc-resistant line and #60, a PrPSc-sensitive cell line. Subsequent infection experiments have shown that this difference in phenotype has been stably maintained in culture for over 1 year (FIG. 3A).

The PrP content, glycosylation pattern and subcellular localisation of the PrP to the lipid rafts was indistinguishable between the two cell lines (FIG. 3B). Moreover experiments using RT-PCR to recover the PrP cDNA from both cell lines revealed that the primary sequence of the protein was identical (data not shown). All these data together demonstrate that differences in phenotype are not simply a result of alterations in the levels of expression of PrP^{C} or mutations within the PrP gene. In conclusion, the difference in the ability of these cells to replicate the prion protein must therefore be due to some other cellular factor.

### Lipid Rafts

Subcellular compartments of lipid rafts were purified from both subclones and individually tested for their converting activity in vitro. Whereas no conversion was seen with lipid rafts from resistant cells (#23), lipid rafts from the sensitive cells (#60) showed significant amplification of PrP^{Sc} (EP03101795.7), suggesting that the presence or absence of factors within lipid rafts, others than PrP^{C}, are likely to be responsible for the different replicating activity.

### Monoclonal Antibodies (Mabs) Generation

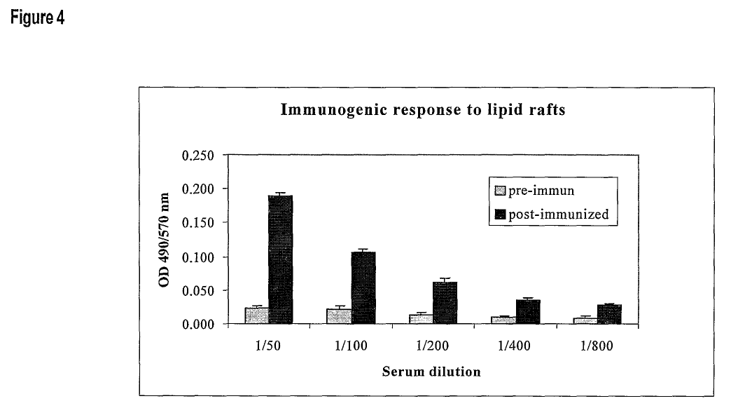

As lipid rafts from clones #23 and #60 show differential ability to convert PrP^{C} to PrP^{Sc}, differences in their composition presumably underlie the difference in converting activity. In the present study, rats were immunized in order to produce antibodies against the components present in these membrane microdomains. Total lipid rafts from both subclones were pooled together in order to include both activating and inhibiting factor(s) that might be involved in the conversion. Rats were injected three times as described in Material and Methods. Samples of blood were taken prior to and after the immunizations and analysed in ELISA for interactions with N2a-lipid rafts. Plates were coated with 100 μl/well of lipid rafts (1 mg total protein) and incubated with several dilutions of serum. Results showed a strong immunogenic response to lipid rafts (FIG. 4).

Lymph nodes were then removed and processed for PEG-mediated fusion with myeloma cells (Sp 2/0) as described in Material and Methods. 631 hybridomas were obtained.

### Primary Screening: ELISA/FACS

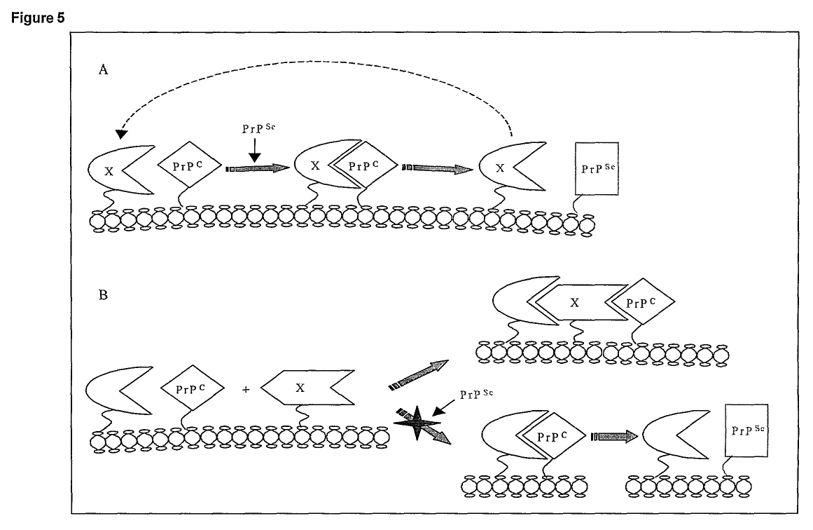

Without wishing to be bound to theory, depending on the nature of the putative conversion factor(s), at least two models can be proposed for prion conversion: either the existence of a factor that promotes conversion in sensitive cells or the presence of an inhibitor in resistant cells (FIG. 5).

With this in mind, a primary screening programme was performed comprising: (i) ELISA in order to detect the Mabs that were specific to lipid rafts, and (ii) FACS analyses against the individual subclones #23 and #60 to monitor whether any of the antigens were differentially expressed on the cell membranes of the two cell types.

(i) ELISA

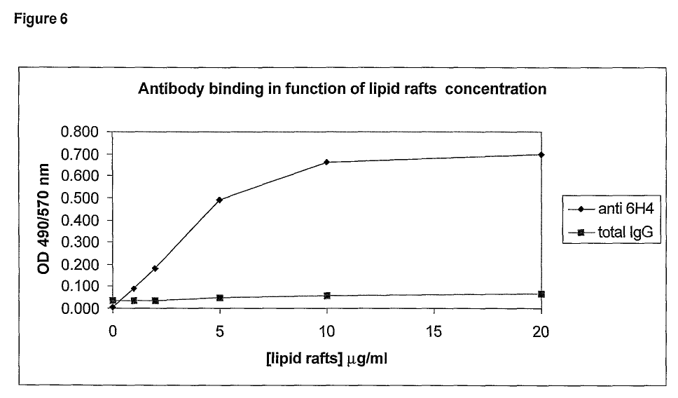

A first ELISA experiment was performed to define the amount of lipid raft protein necessary to saturate the wells. Lipid rafts were first purified from N2a cells (see material and methods), resuspended in coating buffer and coated at different concentrations up to 20 μg/ml. Incubation with the anti-prion Mab, 6H4 followed by a secondary anti-mouse antibody conjugated to HRP demonstrated that a protein concentration of 10 μg/ml (i.e. 1 μg/well) was optimal for the Mab screen (FIG. 6).

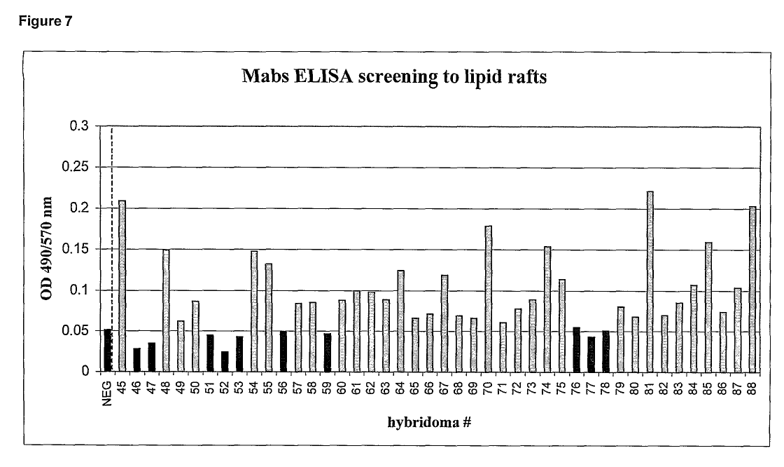

Hybridoma supernatants were then tested against total lipid rafts (1 μg/well). Screening of all the 631 candidates resulted in 195 positives and 436 negatives (representative results are shown in FIG. 7). In addition, all supernatants were screened against recombinant mouse PrP (0.1 μg/well) which showed that none of the Mabs were directed against PrP itself (data not shown).

(ii) FACS

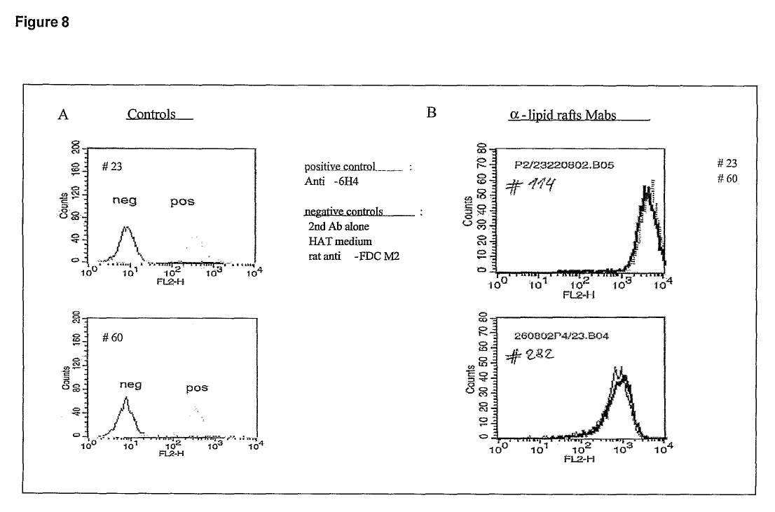

For the FACS analyses resistant and sensitive subclones were incubated separately with the same collection of 631 hybridoma supernatants used above. Representative results are shown in FIG. 8. All positive supernatants in the ELISA screen were also positive by FACS, and interestingly many Mabs considered as negatives by ELISA turned out to be positive in the FACS analysis. Thus the FACS analysis appears to be more sensitive.

Without wishing to be bound to theory, it is suggested that differences in the composition of lipid rafts domains could be responsible for the ability or inability to propagate prions. By producing Mabs against individual components of lipid rafts, it was suggested that by FACS analysis a differential shift for some of them would be observed. However, this was not the case and all Mabs produced similar shifts for both cell lines. Typical results are shown in FIG. 8.

Without whishing to be bound to theory, this lack of differential shift could be explained in several ways: (i) differences in conversion activity may be due to post-translational modifications in the factors involved; (ii) point mutations may occur in residues that are crucial for prion propagation but which cannot be detected by FACS; (iii) conversion factor(s) may be present in insufficient amounts within lipid rafts to produce an immunogenic response.

Since no reliable criteria for selecting among the 464 FACS-positives Mabs were available, it was decided to test all of them in the cell-based secondary screen.

### Secondary Screening: Cell Based Prion Replication Assay

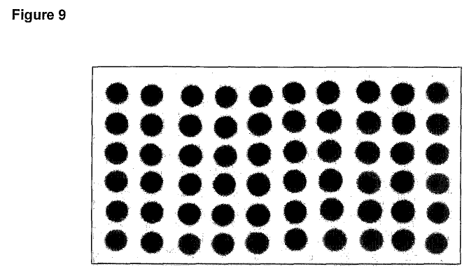

In view of the large numbers of Mabs which came through the primary screening stage, a set up of a high throughput procedure for a cell-based prion replication assay was needed. As a first step, chronically infected N2a (#60) cells were simply grown in 96 well plates in complete DMEM to see whether the scaled down conditions provided a sufficiently robust and reliable signal. To avoid edge effects during cell culture, only the central 60 wells of the 96-well plate were used. The resulting dot-blots shown in FIG. 9 were reassuring and indicated uniform and intense signals, corresponding to the presence of PrP^{Sc} in all wells.

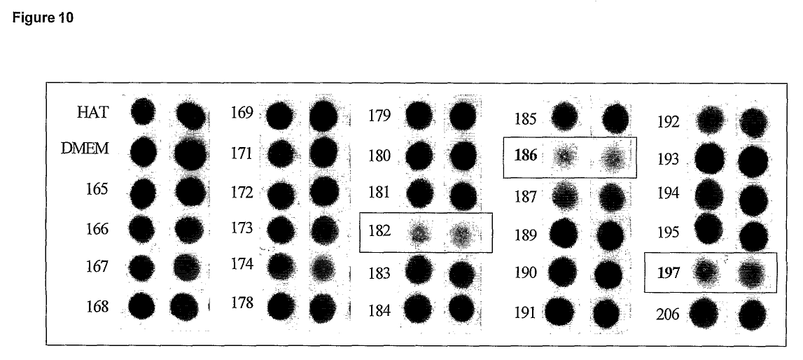

Using the same format, chronically infected N2a cells were cultured in medium containing Mabs (complete DMEM:hybridoma supernatant 1:1) and analysed for PrPSc replication. Effects on growth rates compared to controls were followed under the microscope. Screening of the 464 Mabs confirmed the reliability of the present functional assay. All Mabs were tested in two independent experiments and the resulting cell-blots were almost identical (FIG. 10). From these experiments the Mabs fell into three categories: (1) The majority of antibodies which did not affect the prion replication, (2) a limited number of antibodies which exhibited a significant and reproducible decrease in the PrP^{Sc} signal. These potentially interesting Mabs fell into two sub-classes: (i) 19 Mabs in which the reduced signal was associated with (and probably due to) inhibition of cell growth and (ii) 22 Mabs which interfere with prion replication without affecting the rate of growth. (3) antibodies which appeared to boost prion replication.

Thus in summary, after testing 631 Mabs in an intensive primary screening comprising ELISA and FACS, 464 were selected, and of these 22 (#s 5, 51, 57, 147, 186, 197, 235, 245, 305, 308, 320, 329, 359, 361, 414, 469, 552, 559, 577, 601, 606, 615) seemed to impair the process of prion conversion while 3 (#s 262, 499, 608) seemed to increase PrP conversion. Of the antibodies which had no effect, 3 representatives (#s 93, 122, 306) were selected as negative controls for further studies.

### Discussion

Conversion of the cellular prion protein to the pathological form is the main event underlying TSEs. Whereas PrP^{C} is mainly α-helical, PrP^{Sc} is highly enriched in β-sheets. This drastic change in secondary structure is believed to be assisted by accessory factors often referred as to “protein X”, “factor X” or “conversion factor”. A discontinuous epitope in the PrP protein for factor X binding was proposed (Kaneko et al. 1997) Several PrP^{C}-interacting molecules with a chaperone-like activity have been reported so far: human chaperone proteins BIP and Hsp60 (Jin et al. 2000 and Edenhofer et al. 1996), bacterial and yeast chaperones GroEL protein and the heat shock protein (hsp) 104 respectively (DebBurman et al. 1997). Other molecules such as sulfated glycosaminoglycans (GAGs) (Brinacombe et al. 1999), neuronal adhesion molecules (N-CAMs) (Schmitt et al. 2001), laminin and its related receptor (Martins et al. 2002) as well as nucleic acids (Nandi et al. 2002 and Cordeiro et al. 2001) were shown to bind PrP^{C}. However there is no evidence to date that any of these proteins are factors responsible for prion conversion.

By producing monoclonal antibodies against lipid rafts from cells possessing all the factors required for prion conversion, a small subset of Mabs which interfere with prion replication in chronically infected N2a (ScN2a) was identified. Several antibodies have previously been reported to abolish infection in cells but all of them were directed against different epitopes of the prion protein (Peretz et al. 2001). In contrast, ELISA screening against recombinant mouse PrP showed that none of the present Mabs are directed against PrP^{C}, suggesting that in the present case prion replication is impaired through the binding of Mabs to other factors involved in the process of conversion. The fact that Mabs can “cure” ScN2a argues in favour of a positive acting factor (FIG. 5A) present in sensitive cells.

Purification and subsequent identification of the antigen(s) that Mabs are directed to will help our understanding of the conversion process, while further characterization of the antigens, for example by generating knock-out animals for these putative factors would also help in the understanding of their normal physiological role in non-pathological situations. Finally, the use of Mabs that inhibit prion replication could provide a potential therapy for prion diseases.

### Example 2

### Introduction

Experiments were performed to characterise further the most interesting antibodies described in example 1. The original frozen hybridoma stocks were re-cultured on a larger scale and secreted antibodies were purified and concentrated from the culture supernatant (see Methods). Some clones grew poorly. Of the original 22 inhibitory clones (#s 5, 51, 57, 197, 235, 245, 305, 308, 320, 329, 615) grew well and were characterized further. The effect of the purified antibodies from these clones on PrP replication is described below. Antibodies from several clones which, from the experiments in example 1 were found to have no effect on PrP replication (#s 93, 122, 306), were included as negative controls.

### Material and Methods

### Purification and Concentration of Rat Monoclonal Antibodies

Hybridomas were grown in 10 cm culture dishes in ultra-low IgG medium. To purify antibodies, 2.7 ml of hybridoma supernatant, was mixed with 300 μl of Tris-HCl 1M, pH 7.5 and 0.527 g of NaCl to give a final concentration: 0.1M Tris-HCl pH 7.5, 3M NaCl. The high salt concentration is used to increase the affinity of rat IgG to protein G. Each supernatant was then mixed with 100 μl Protein-G GammaBind Plus Sepharose beads (Pharmacia) equilibrated with the same buffer and incubated overnight at 4° C. with gentle agitation. The beads were then transferred to a disposable column, washed with at least 10 column volumes of 0.1M Tris-HCl pH 7.5 and Mabs were eluted with 0.1M Glycine pH 2.5 (2 drop fractions collected). The eluted proteins were collected directly into 12 μl 1M Tris-HCl pH 8.0 to restore neutral pH. Fractions containing IgGs were identified by Coomassie blue staining or by Western blotting with HRP-coupled anti-rat antibodies. Appropriate fractions were pooled and concentrated using Centricon YM 30 spin columns (Millipore) according to the makers instructions. Protein concentration was determined using the Bradford method (Biorad).

### Results

Employing the methodology and hybridomas described in example 1, and by performing additional experiments based on studies with the crude hybridomas supernatants, three classes of antibodies could be identified:

1) Mabs which appeared to inhibit prion replication without affecting cell growth (identified as #s 5, 51, 57, 197, 235, 245, 305, 308, 320, 329, 615)

2) Mabs which appeared to boost prion replication (identified as #s 262, 499, 608), and

3) the majority of Mabs, which had no effect on prion replication (of which #s 93, 122, 306, were selected as representatives).

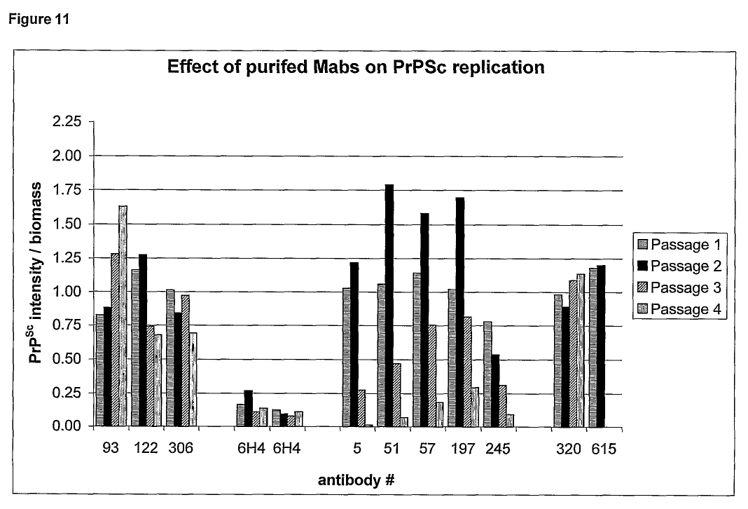

This example (FIG. 11) provides the final results of the effect of Mabs on PrP^{Sc} replication. All Mabs were purified from the culture supernatants and used at a final concentration of 2 μg/ml. Culture dot blotting and quantitation of the PrPSc levels was performed as described in Example 1. The data show that 6H4 is a powerful inhibitor of PrP^{Sc} replication, confirming results already in the literature, and that the purified antibodies from the hybridomas previously defined as negative controls do not affect PrP^{Sc} replication. Of the antibodies previously defined as inhibitory, 5 of them (#s 5, 51, 57, 197, and 245) showed clear inhibition of PrP replication after several passages, 2 of them #s 320 and 615) appeared to have lost the inhibitory effect, while the remainder (#s 235, 305, 308, 329) upon careful retesting using the quanatitative dot blotting procedure proved to be inhibitory to cell growth which thus explains the reduced signal. Some of the hybridoma clones are deposited at the European Collection of Cell Cultures (ECACC, http://www.ecacc.org.uk/). The hybridoma clone designated #51 is deposited at the ECACC under Provisional Accession No. 05021601. The hybridoma clone designated #57 is deposited at the ECACC under Provisional Accession No 05030901. The hybridoma clone designated #245 is deposited at the ECACC under Provisional Accession No. 05021603.

### Discussion

Employing the above procedures, it was possible to select antibodies that could modulate prion replication. Hybridomas that allow for the selection of antibodies able to modulate conversion of PrP^{C} into PrP^{Sc} were therefore generated. More particularly, hybridomas that allow selection of antibodies able to prevent or favour conversion of PrP^{C} into PrP^{Sc} were obtained. The hybridoma clones designated #5, #51, #57, #197 and #245 therefore allow for the selection of antibodies able to prevent conversion of PrP^{C} into PrP^{Sc}, whereas the hybridoma clones designated #262, #499 and #608 therefore allow for the selection of antibodies able to favour conversion of PrP^{C} into PrP^{Sc}.

The antigens recognised by the above antibodies can be obtained by conventional techniques; e.g.:

1) repurifying the hybridomas by single cell cloning and then rescreening for the biological activity using the purified clones;

2) using the purified antibodies to isolate the cognate antigen from the total lipid raft fraction;

3) cloning of the cDNA corresponding to the antigen and expression of the recombinant protein;

4) investigating the biological activity of the recombinant protein in relation to prion replication;

5) identifying, cloning and characterizing the human equivalent.

The antigens derived from the above Mabs are therefore either able to prevent or increase conversion of PrP^{C} into PrP^{Sc}.

The antigens recognised by the hybridoma clones are identified either as conversion factors (one of the factors implicated in prion replication, e.g. as the one identified in EP03101795.7, i.e. ApoB), in their ability to favour conversion of PrP^{C} into PrP^{Sc}, or as inhibitors of prion replication, in their ability to prevent conversion of PrP^{C} into PrP^{Sc}.

The selected antibodies from the hybridoma clones designated #5, #51, #57, #197 and #245 are therefore either antagonistic antibodies towards positive acting factors in prion replication or agonistic antibodies towards negative acting factors.

The antigens, derived from the hybridoma clones designated #262, #499 and #608, are also identified here as either conversion factors (one of the factors implicated in prion replication, e.g. as the one identified in EP03101795.7, i.e. ApoB), in their ability to favour conversion of PrP^{C} into PrP^{Sc}, or as inhibitors of prion replication, in their ability to prevent conversion of PrP^{C} into PrP^{Sc}. But here, the selected antibodies from the hybridoma clones designated #262, #499 and #608 are either agonistic antibodies of conversion factors or antagonistic antibodies of inhibitors of prion replication.

In summary, conversion factors or inhibitors of prion replication are obtained by the methods of the invention.

The use of inhibitors of prion replication or specific parts thereof and/or antibodies or fragments thereof targeted to conversion factors seems particularly suited for the treatment of a conformational disease and particularly for prion diseases. These inhibitors of prion replication or/and antibodies targeted to conversion factors could be combined with other known inhibitors of prion replication (e.g. see above background section or antibodies targeted to PrP^{Sc} itself like 6H4) or/and known antibodies targeted to conversion factors (e.g. identified in EP03101795.7).

### REFERENCES

Aizawa et al., Brain R. 768 (1-2), 208-14, 1997;
Baron et al., The EMBO Journal, 21, 5,1031-1040, 2002;
Bosque, P. J. & Prusiner, S. B. Journal of Virology. 74, 4377-4386, 2000;
Brimacombe, D. B., Bennett, A. D., Wusteman, F. S., Gill, A. C., Dann, J. C. & Bostock, C. J. Biochemical Journal 342, 605-613, 1999;
Bruce et al., Nature, 389, 498-501, 1997;
Bueler et al., Cell 73, 1339-1347, 1993;
Butler, D. A., Scott, M. R., Bockman, J. M., Borchelt, D. R., Taraboulos, A., Hsiao, K. K., Kingsbury, D. T. & Prusiner, S. B. Journal of Virology. 62, 1558-1564, 1988;
Chabry et al., J. Biol. Chem. 273, 13203-13207, 1998;
Choi et al., J. Lip. Res., 38(1)77-85, 1997;
Cohen et al., Ann. Rev. Biochem 67,793-819, 1998;
Cordeiro, Y., Machado, F., Juliano, L., Aparecida Juliano, M., Brentani, R. R., Foguel, D. & Silva, J. L. Journal of Biological Chemistry 276, 49400-49409, 2001;
DebBurman, S. K., Raymond, G. J., Caughey, B. & Lindquist, S. Proceedings of the National Academy of Sciences of the United States of America. 94, 13938-13943, 1997;
Edenhofer, F., Rieger, R., Famulok, M., Wendler, W., Weiss, S. & Winnacker, E. L. Journal of Virology 70, 4724-4728, 1996;
Enari et al., Proc. Natl. Acad. Sci. USA 98, 9295-9299, 2001;
Fantini et al., Expert Reviews in Molecular Medicine, Dec. 20, 1-22, 2002;
Fivaz, M., Vilbois, F., Pasquali, C. & van, d. Electrophoresis. 21, 3351-3356, 2000;
Hooper et al., Mol. Memb. Biol. 16, 145-156, 1999;
Jin, T., Gu, Y., Zanusso, G., Sy, M., Kumar, A., Cohen, M., Gambetti, P. & Singh, N. Journal of Biological Chemistry 275, 38699-38704, 2000.
Kaneko, K., Zulianello, L., Scott, M., Cooper, C. M., Wallace, A. C., James, T. L., Cohen, F. E. & Prusiner, S. B. Proceedings of the National Academy of Sciences of the United States of America. 94, 10069-10074, 1997;
Lehninger et al., Principles of Biochemistry, 2nd Ed. New York: Worth Publishers, 1993;
Martins, V. R., Linden, R., Prado, M. A., Walz, R., Sakamoto, A. C., Izquierdo, I. & Brentani, R. R. FEBS Letters. 512, 25-28, 2002;
Nandi, P. K., Leclerc, E., Nicole, J. C. & Takahashi, M. Journal of Molecular Biology. 322, 153-161, 2002;
Pan et al., Proc. Natl. Acad. Sci. (USA) 90, 10962-10966, 1993;
Peretz, D., Williamson, R. A., Kaneko, K., Vergara, J., Leclerc, E., Schmitt-Ulms, G., Mehlhorn, I. R., Legname, G., Wormald, M. R., Rudd, P. M. et al. Nature 412, 739-743, 2001;
Prusiner, Science 252, 1515-1522, 1991;
Prusiner, Proc. Natl. Acad. Sci. USA 95, 13363-13383, 1998;
Roos et al., Brain 96, 1-20, 1973;
Saborio et al., Biochem. Biophys. Res. Commun. 258, 470-475, 1999;
Saborio et al., Nature 411, 810-813, 2001;
Segrest et al., Journal of Lipid Research, 42, 1346-1367, 2001;
Scott et al., Proc. Natl. Acad. Sci. USA 96, 15137-15142, 1999;
Schmitt-Ulms, G., Legname, G., Baldwin, M. A., Ball, H. L., Bradon, N., Bosque, P. J., Crossin, K. L., Edelman, G. M., DeArmond, S. J., Cohen, F. E. et al. Journal of Molecular Biology 314, 1209-1225, 2001;
Simons et al, Molecular Cell Biology 1, p 31-41, 2000;
Soto et al., Trends Mol. Med. 7, 109-114, 2001;
Taraboulos at al., The Journal of Cell Biology, 129 (1), 121-132, 1995;
Telling et al., Proc. Natl. Acad. Sci. USA 91, 9936-9940, 1994;
Tsui-Pierchala et al., Trends Neurosci. 25, 412-417, 2002;
Wang et al., Aeterioscler. Thromb. Vas. Biol., 20(5), 1301-8, 2000;
Will et al., Lancet 347, 925, 1996;
Yamada et al, Ann Clin. Lab. Sci. 27(4), 77-85, 1997;
U.S. Pat. No. 5,276,059
U.S. Pat. No. 5,134,121
U.S. Pat. No. 6,355,610
U.S. Pat. No. 5,948,763
U.S. Pat. No. 6,552,922
U.S. Pat. No. 6,197,972
US 20020128175
WO 0168710
WO 0204954
WO 03002533
WO 03045921

1-27. (canceled)

28. A method for generating an antibody against a lipid raft target associated with a type of PrP^{Sc} cells, comprising:
a. isolating said lipid rafts from said type of PrPSc cells; and
b. immunizing an animal host by said isolated lipid rafts.

29. The method according to claim 28, wherein said type of PrP^{Sc} cells are either PrP^{Sc} sensitive cells or PrP^{Sc} resistant cells.

30. The method according to claim 28 further comprising:
c. producing hybridomas from the immunized animal host, wherein said hybridomas produce monoclonal antibodies;
d. selecting said monoclonal antibodies; and
e. purifying said selected monoclonal antibodies.

31. The method according to claim 30, wherein said selecting further comprises selecting monoclonal antibodies that modulate conversion of PrP^{C} into PrP^{Sc} of said type of PrP^{Sc} sensitive cells.

32. The method according to claim 29, wherein said type of PrP^{Sc} sensitive cells are neuroblastoma cells.

33. The method according to claim 32, wherein said type of neuroblastoma cells are scN2a or N2A cells.

34. A method of identifying a lipid raft target comprising identifying an antigen that binds to antibodies that bind isolated lipid rafts from a type of PrP^{Sc} cells and determining a partial or full amino acid sequence or nucleic acid sequence of said antigen.

35. A composition of matter comprising:
(a) a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts; and
(iii) producing hybridomas from the immunized animal host, wherein said hybridomas produce monoclonal antibodies;
(b) the hybridoma clone designated #51 deposited at the ECACC under Provisional Accession No. 05021601;
(c) the hybridoma clone designated #57 deposited at the ECACC under Provisional Accession No. 05030901;
(d) the hybridoma clone designated #245 deposited at the ECACC under Provisional Accession No. 05021603;
(e) an isolated antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(f) the monoclonal antibody generated by hybridoma clone designated #51 deposited at the ECACC under Provisional Accession No. 05021601;
(g) the monoclonal antibody generated by hybridoma clone designated #57 deposited at the ECACC under Provisional Accession No. 05030901;
(h) the monoclonal antibody generated by hybridoma clone designated #245 deposited at the ECACC under Provisional Accession No. 05021603;
(i) an antigen, or specific portion thereof, that binds to:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901; or
(D) the monoclonal antibody generated by hybridoma clone designated #245 deposited at the ECACC under Provisional Accession No. 05021603;
(j) an isolated antibody, monoclonal antibody, chimeric antibody, fully humanized antibody, anti-anti-ID antibody or fragment thereof being capable of specifically binding an antigen that binds to:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901; or
(D) the monoclonal antibody generated by hybridoma clone designated #245 deposited at the ECACC under Provisional Accession No. 05021603; or
(k) a pharmaceutical composition comprising a pharmaceutically acceptable carrier and:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901;
(D) the monoclonal antibody generated by hybridoma clone designated #245 deposited at the ECACC under Provisional Accession No. 05021603; or
(E) an isolated antibody, monoclonal antibody, chimeric antibody, fully humanized antibody, anti-anti-ID antibody or fragment thereof being capable of specifically binding an antigen that binds to:
(a) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(b) the monoclonal antibody generated by hybridoma clone designated #51 deposited at the ECACC under Provisional Accession No. 05021601;
(c) the monoclonal antibody generated by hybridoma clone designated #57 deposited at the ECACC under Provisional Accession No. 05030901; or
(d) the monoclonal antibody generated by hybridoma clone designated #245 deposited at the ECACC under Provisional Accession No. 05021603.

36. The composition of matter according to claim 35, wherein said hybridoma produces monoclonal antibodies that modulate conversion of PrP^{C} into PrP^{Sc} of said type of PrP^{Sc} sensitive cells.

37. The composition of matter according to claim 35, wherein said hybridoma is produced by immunizing an animal host with neuroblastoma cells.

38. The composition of matter according to claim 37, wherein said hybridoma is produced by immunizing an animal host with neuroblastoma cells selected from scN2a or N2A cells.

39. The composition of matter according to claim 35, wherein said pharmaceutical composition comprises an antibody or antibody fragment is further capable of regulating a biochemical activity of said antigen or a specific portion thereof.

40. A method of treating a conformational disease comprising the administration of an effective amount of a composition comprising an antibody to an individual having said conformational disease, wherein said antibody is:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901;
(D) the monoclonal antibody generated by hybridoma clone designated #245 deposited at the ECACC under Provisional Accession No. 05021603; or
(E) an isolated antibody, monoclonal antibody, chimeric antibody, fully humanized antibody, anti-anti-ID antibody or fragment thereof being capable of specifically binding an antigen that binds to:
(a) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(b) the monoclonal antibody generated by hybridoma clone designated #51 deposited at the ECACC under Provisional Accession No. 05021601;
(c) the monoclonal antibody generated by hybridoma clone designated #57 deposited at the ECACC under Provisional Accession No. 05030901; or
(d) the monoclonal antibody generated by hybridoma clone designated #245 deposited at the ECACC under Provisional Accession No. 05021603.

41. The method according to claim 40, wherein said conformational disease is a prion disease, Alzheimer's Disease, amyotrophic lateral sclerosis (ALS), Pick's disease, Parkinson's disease, Frontotemporal dementia, Diabetes Type II, Multiple myeloma, Plasma cell dyscrasias, Familial amyloidotic polyneuropathy, Medullary carcinoma of thyroid, Chronic renal failure, Congestive heart failure, Senile cardiac and systemic amyloidosis, Chronic inflammation, Atherosclerosis, Familial amyloidosis Gelsolin and Huntington's disease, cerebral amyloid angiopathy (CAA).

42. A method for the detection of PrP^{Sc} within a sample, which assay comprises (i) contacting said sample with a antigen, or a specific portion thereof, or a monoclonal antibody, antibody or antigen binding fragment thereof; (ii) contacting sample obtained in (i) with PrP^{C} or PrP^{C} containing mixtures; and (iii) determining the presence and/or amount of PrP^{Sc} in said sample, wherein said antigen, or specific portion thereof, binds to:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901;
(D) the monoclonal antibody generated by hybridoma clone designated #245 deposited at the ECACC under Provisional Accession No. 05021603; or
(E) an isolated antibody, monoclonal antibody, chimeric antibody, fully humanized antibody, anti-anti-ID antibody or fragment thereof being capable of specifically binding an antigen that binds to:
(a) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(b) the monoclonal antibody generated by hybridoma clone designated #51 deposited at the ECACC under Provisional Accession No. 05021601;
(c) the monoclonal antibody generated by hybridoma clone designated #57 deposited at the ECACC under Provisional Accession No. 05030901; or
(d) the monoclonal antibody generated by hybridoma clone designated #245 deposited at the ECACC under Provisional Accession No. 05021603; or
wherein said antibody is:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901;
(D) the monoclonal antibody generated by hybridoma clone designated #245 deposited at the ECACC under Provisional Accession No. 05021603; or
(E) an isolated antibody, monoclonal antibody, chimeric antibody, fully humanized antibody, anti-anti-ID antibody or fragment thereof being capable of specifically binding an antigen that binds to:
(a) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(b) the monoclonal antibody generated by hybridoma clone designated #51 deposited at the ECACC under Provisional Accession No. 05021601;
(c) the monoclonal antibody generated by hybridoma clone designated #57 deposited at the ECACC under Provisional Accession No. 05030901; or
(d) the monoclonal antibody generated by hybridoma clone designated #245 deposited at the ECACC under Provisional Accession No. 05021603.

43. The method according to claim 42, wherein said sample comprises tissue extracted from an animal that has died and said method comprises contacting the tissue with an antibody or an antigen binding fragment thereof and determining if the antibody has bound to said antigen or a specific portion thereof; wherein presence of said antigen or a specific portion thereof in the tissue is indicative of PrP^{Sc} infection and wherein said antibody is:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901;
(D) the monoclonal antibody generated by hybridoma clone designated #245 deposited at the ECACC under Provisional Accession No. 05021603; or
(E) an isolated antibody, monoclonal antibody, chimeric antibody, fully humanized antibody, anti-anti-ID antibody or fragment thereof being capable of specifically binding an antigen that binds to:
(a) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(b) the monoclonal antibody generated by hybridoma clone designated #51 deposited at the ECACC under Provisional Accession No. 05021601;
(c) the monoclonal antibody generated by hybridoma clone designated #57 deposited at the ECACC under Provisional Accession No. 05030901; or
(d) the monoclonal antibody generated by hybridoma clone designated #245 deposited at the ECACC under Provisional Accession No. 05021603.

44. A method for identifying a compound which modulates the transition of PrP^{C} into PrP^{Sc} comprising:
(i) contacting said sample with an antigen or a specific portion thereof or with an antibody or an antigen binding fragment thereof and at least another conversion factor (a) in the presence of said modulatory compound and (b) in the absence of said compound;
(ii) contacting the mixtures obtained in step (i) a and (i) b with PrP^{C} or PrP^{C} containing mixtures; and
(iii) determining the amount of PrP^{Sc} (a) in the presence of said modulatory compound and (b) in the absence of said modulatory compound;
wherein said antigen, or specific portion thereof, binds to:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901;
(D) the monoclonal antibody generated by hybridoma clone designated #245 deposited at the ECACC under Provisional Accession No. 05021603; or
(E) an isolated antibody, monoclonal antibody, chimeric antibody, fully humanized antibody, anti-anti-ID antibody or fragment thereof being capable of specifically binding an antigen that binds to:
(a) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(b) the monoclonal antibody generated by hybridoma clone designated #51 deposited at the ECACC under Provisional Accession No. 05021601;
(c) the monoclonal antibody generated by hybridoma clone designated #57 deposited at the ECACC under Provisional Accession No. 05030901; or
(d) the monoclonal antibody generated by hybridoma clone designated #245 deposited at the ECACC under Provisional Accession No. 05021603; or
wherein said antibody is:
(A) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(B) the monoclonal antibody generated by hybridoma clone designated #51 deposited at the ECACC under Provisional Accession No. 05021601;
(C) the monoclonal antibody generated by hybridoma clone designated #57 deposited at the ECACC under Provisional Accession No. 05030901;
(D) the monoclonal antibody generated by hybridoma clone designated #245 deposited at the ECACC under Provisional Accession No. 05021603; or
(E) an isolated antibody, monoclonal antibody, chimeric antibody, fully humanized antibody, anti-anti-ID antibody or fragment thereof being capable of specifically binding an antigen that binds to:
(a) an antibody or antigen binding fragment thereof generated by a hybridoma produced by method for generating an antibody against a lipid raft target associated with a type of PrP^{Sc} cells, comprising:
(i) isolating said lipid rafts from said type of PrP^{Sc} cells;
(ii) immunizing an animal host by said isolated lipid rafts;
(iii) producing hybridomas from the immunized animal host; and
(iv) isolating the antibody produced by said hybridoma;
(b) the monoclonal antibody generated by hybridoma clone designated #51 deposited at the ECACC under Provisional Accession No. 05021601;
(c) the monoclonal antibody generated by hybridoma clone designated #57 deposited at the ECACC under Provisional Accession No. 05030901; or
(d) the monoclonal antibody generated by hybridoma clone designated #245 deposited at the ECACC under Provisional Accession No. 05021603.

45. The method according to claim 44, wherein said conversion factor is Apolipoprotein B or a fragment thereof.
